(19) United States
(12) Patent Application Publication
(10) Pub. No.: US 2011/0256169 A1
(43) Pub. Date: Oct. 20, 2011

(54) NOVEL POLYPEPTIDES RELATED TO B-TYPE NATRIURETIC PEPTIDES AND METHODS OF THEIR IDENTIFICATION AND USE

(75) Inventors: Koen Kas, Schilde (BE); Robin Tuytten, Ekeren (BE); Koen De Cremer, Lede (BE); Griet Vanpoucke, Merelbeke (BE)

(73) Assignee: Pronota N.V., Zwijnaarde (BE)

(21) Appl. No.: 12/991,869

(22) PCT Filed: May 14, 2009
(86) PCT No.: PCT/EP09/55851

§ 371 Date: Nov. 9, 2010

(30) Foreign Application Priority Data
May 16, 2008 (EP) 08156351.2
Nov. 3, 2008 (EP) 08168196.7

(51) Int. Cl. A61K 39/00; C12P 21/06; C40B 30/04; G01N 33/566; G01N 30/02; C07K 16/26; C07K 1/14; A61P 37/04; H01J 49/26; C07K 14/575; C07K 2/00

(52) U.S. Cl. 424/195.11; 530/399; 530/324; 435/68.1; 506/9; 436/501; 530/350; 530/300; 530/387.9; 424/198.1; 250/282; 73/61.52

(57) ABSTRACT

The invention provides novel fragments of proBNP and NTproBNP, particularly useful in prognosing or diagnosing acute heart failure, chronic heart failure or sepsis.

### FIELD OF THE INVENTION

The invention relates to protein and/or peptide based biomarkers and molecules specifically binding thereto for use in diagnosis, prognosis and prediction of disease or determination of a particular condition in a subject. In particular, certain peptides or proteins as biomarkers for acute heart failure, chronic heart failure or sepsis and methods for use of the same in diagnosis, prognosis and/or prediction of the onset of said conditions including methods involving determining increased, decreased or altered expression of said biomarkers in a sample of a subject are encompassed in the invention. More in particular, the invention concerns biomarkers related to pro-B-type natriuretic peptide (proBNP) and amino terminal pro-B-type natriuretic peptide (NTproBNP).

### BACKGROUND OF THE INVENTION

In many diseases and conditions, a positive outcome of treatment and/or prophylaxis is strongly correlated with early and/or accurate diagnosis of the disease or condition. However, often there are no effective methods of early diagnosis and treatments are therefore often administered too late, inappropriately or to individuals who will not benefit from it. As a result, many drugs that may be beneficial for some patients may work poorly, not at all, or with adverse effect in other patients. Thus, there is a need for innovative strategies that will allow early detection, prediction, prognosis, diagnosis and treatment of diseases and other biological conditions. There is also a need to determine the ability, or inability, of a patient to tolerate medications or treatments.

Heart failure is a major public health issue in developed countries and is the cause of considerable morbidity and mortality among older adults. It is usually a chronic disease characterised by frequent recurrent decompensation leading to worsening breathing problems. Moreover, 5 years after diagnosis 50% of heart failure patients will have died from the disease.

Acute decompressed heart failure (AHF) is a sudden inability of the heart to pump efficiently and where it can no longer foresee the bodily demands for oxygen. 90% of AHF admissions are from patients with chronic heart disease, the remaining 10% are de novo patients. The clinical signs of heart disease and AHF are often non-specific which makes unambiguous diagnosis often very difficult.

The current biomarker used for diagnosing AHF in an emergency setting is B-type natriuretic peptide (BNP). In an emergency setting BNP at a cut-off value of 100 pg/ml has a sensitivity of 90% and a specificity of 73%. Although this marker is highly sensitive, the specificity is relatively low and is especially problematic in the grey zone between 100-400 pg/ml. Also, BNP levels vary with age, sex, weight and other medical conditions and its levels are elevated in patients with CHF. Especially in predicting recovery of sepsis or heart failure patients, the current BNP measurement tools are insufficiently reliable.

There is accordingly medical need for further markers for AHF that may complement BNP. Clerico et al. 2007 (Clin Chem 3: 813-22) reported that N-terminal part of proBNP (NTproBNP) assays also have a high degree of diagnostic accuracy and clinical relevance for both acute and chronic heart failure.

Accordingly, there exists a need for precise and more detailed understanding of the biology of NTproBNP, and concurrently for improved NTproBNP and proBNP assays particularly providing for increased information contents, accuracy or specificity.

Sepsis is more commonly called a blood stream infection or blood poisoning. It is the presence of bacteria (bacteremia), infectious organisms, or their toxins in the blood or other tissues of the body. Sepsis often occurs in patients suffering from systemic inflammatory response syndrome (SIRS), as a result of e.g. surgery, trauma, burns, pancreatitis and other non-infectious events that cause inflammation to occur. SIRS combined with an infection is called sepsis and can occur in many different stages of severity. The infection can occur simultaneously with the occurrence of SIRS e.g. due to infection of a wound or trauma or can occur later due to the latent presence of an infectious organism. Sepsis may be associated with clinical symptoms of systemic (body wide) illness, such as fever, chills, malaise, low blood pressure, and mental status changes. Sepsis can be a serious situation, a life threatening disease calling for urgent and comprehensive care. Treatment depends on the type of infection, but usually begins with antibiotics or similar medications.

As sepsis may be the result of infection by a wide variety of organisms it is a condition which is particularly difficult to predict and diagnose early enough for effective intervention. It is an excessive and uncontrolled inflammatory response in an individual usually resulting from an individual's inappropriate immune system response to a pathogenic organism. Moreover, there may not be significant numbers of organisms at accessible sites or in body fluids of the affected individual, thus increasing the difficulty of diagnosis. There is therefore a need to identify biomarkers indicating the risk, or early onset of sepsis, regardless of the causative agent, to allow early and effective intervention.

The present invention addresses the above needs in the art by identifying further novel biomarkers related to proBNP and NTproBNP and provides more reliable methods of diagnosing, predicting or prognosing diseases or disorders for which measuring of BNP-protein processing is relevant such as acute heart failure (AHF), chronic heart failure (CHF) or sepsis.

### SUMMARY OF THE INVENTION

The inventors have recognised previously unknown processing or proteolysis occurring near the amino-terminus of proBNP and/or NTproBNP. In particular, the inventors have revealed the existence of novel fragments of proBNP and/or NTproBNP in samples from subjects, which start at amino acid positions 3, 4 or 7 of proBNP. The inventors are the first to enable detection of these 3 truncated fragments in samples from patients and to establish their respective values in prognosis, diagnosis and prediction of diseases for which measuring of BNP-protein processing is relevant such as acute heart failure (AHF), chronic heart failure (CHF) or sepsis.

Previous assays for detection of proBNP and/or NTproBNP in samples were unaware of and thus did not take account of said heterogeneity at the N-terminus of proBNP and NTproBNP. Such assays were therefore bound to yield incomplete or even incorrect data, but in any instance data lacking some useful information content. For example, known assays use antibody reagents recognising epitopes that involve the starting amino acids of proBNP. These assays may not adequately detect the now discovered N-terminally truncated proBNP and/or NTproBNP peptides, causing under- or overestimation of the actual amount of proBNP and/or NTproBNP derived analytes in samples. In addition, the various forms of proBNP or NTproBNP peptides may have distinct properties or significance as biomarkers for pathological conditions. Therefore, it is advantageous to maximise the information obtained about a sample by detecting an increased diversity of said proBNP or NTproBNP forms.

Hence, in an aspect the invention provides an isolated fragment of pro-B-type natriuretic peptide (proBNP) selected from proBNP 3-108, 4-108 and 7-108, or a C-terminally truncated form of any one thereof.

In another aspect is provided an isolated fragment of amino terminal pro-B-type natriuretic peptide (NTproBNP) selected from NTproBNP 3-76, 4-76 and 7-76, or a C-terminally truncated form of any one thereof.

The proBNP or NTproBNP, and thus the herein disclosed isolated fragments and C-terminally truncated forms thereof, may preferably be human.

The C-terminally truncated forms of the herein disclosed fragments of proBNP or NTproBNP may typically arise through exo- and/or endoproteolysis of the respective fragments, such as for instance through chemical, physical or enzymatic proteolysis of the respective fragments. For example, said C-terminally truncated forms can arise due to partial degradation, proteolytic processing or cleavage of the respective fragments in vivo, in vitro, in a biological sample, in a separated fraction of the biological sample, or subsequent to isolation of the fragments. By means of example, enzymatic proteolysis of the respective proBNP or NTproBNP fragments by one or more exo- and/or endoproteinases can yield C-terminally truncated forms of said fragments as meant herein. C-terminally truncated forms of the herein disclosed fragments of proBNP or NTproBNP present in biological samples may give useful information about the presence and/or quantity of the respective fragments in said samples, whereby the detection of such C-terminally truncated forms can be of interest.

In a further embodiment, C-terminally truncated forms of the herein disclosed fragments of proBNP or NTproBNP may be obtainable or directly obtained by endoproteinase digest of the respective proBNP or NTproBNP fragments. Such digests can generate readily detectable C-terminally truncated forms that are representative of, and thereby allow measuring the presence and/or quantity of, any longer forms of the proBNP or NTproBNP fragments from which they derive. Preferably, the endoproteinase digest may be by trypsin, which shows high specificity and efficiency of cleavage and thereby assures reproducible truncation of the peptides.

For example, the C-terminally truncated forms of the herein disclosed fragments of proBNP or NTproBNP may be chosen from proBNP or NTproBNP 3-21, 4-21 and 7-21. Such forms can inter alia arise from trypsin digest of any longer forms of the proBNP or NTproBNP fragments and can thus advantageously represent, and allow measuring the presence and/or quantity of, said longer forms of the proBNP or NTproBNP fragments from which they derive.

The herein disclosed fragments of proBNP or NTproBNP and C-terminally truncated forms thereof are useful biomarkers.

A further aspect thus provides a method for assaying proBNP in a sample, comprising specifically measuring the presence and/or quantity in said sample of one or more fragments of proBNP selected from proBNP 3-108, 4-108 and 7-108 and C-terminally truncated forms thereof. The proBNP assay may also measure the presence and/or quantity in the sample of any other forms of proBNP, such as in particular of proBNP 1-108 and C-terminally truncated forms thereof.

Another aspect provides a method for assaying NTproBNP in a sample, comprising specifically measuring the presence and/or quantity in said sample of one or more fragments of NTproBNP selected from NTproBNP 3-76, 4-76 and 7-76 and C-terminally truncated forms thereof. The NTproBNP assay may also measure the presence and/or quantity in the sample of any other forms of NTproBNP, such as in particular of NTproBNP 1-76 and C-terminally truncated forms thereof.

By means of example, some measurement methods may discriminate the different proBNP and/or NTproBNP peptides at the N-terminus, without determining the C-terminus of such fragments. Hence, the above aspects also include methods for assaying proBNP and NTproBNP in a sample, comprising specifically measuring the presence and/or quantity in said sample of one or more fragments of proBNP and NTproBNP selected from proBNP 3-108 and NTproBNP 3-76, proBNP 4-108 and NTproBNP 4-76, or proBNP 7-108 and NTproBNP 7-76, and C-terminally truncated forms thereof. Such assays may also measure the presence and/or quantity in the sample of any other forms of proBNP and NTproBNP, such as in particular of proBNP 1-108 and NTproBNP 1-76 and C-terminally truncated forms thereof. Other assays may discriminate the present proBNP and/or NTproBNP fragments and C-terminally truncated forms thereof also on the basis of their C-terminus.

In an embodiment, the proBNP or NTproBNP, and thus the herein measured fragments and C-terminally truncated forms thereof, may be human.

In a further embodiment, the present methods for assaying proBNP or NTproBNP are immunoassay methods, mass spectrometry analysis methods or chromatography methods, or a combination thereof.

Conditions and diseases in which the herein disclosed fragments of proBNP or NTproBNP and C-terminally truncated forms thereof are useful as biomarkers include in particular acute heart failure (AHF), chronic heart failure (CHF) and sepsis.

Hence, a further aspect provides a method for the prediction, prognosis and/or diagnosis of AHF, CHF or sepsis in a subject, comprising:
(a) assaying proBNP in a sample from the subject, comprising specifically measuring the presence and/or quantity in said sample of one or more fragments of proBNP selected from proBNP 3-108, 4-108 and 7-108 and C-terminally truncated forms thereof;
(b) comparing the presence and/or quantity of said one or more fragments of proBNP and C-terminally truncated forms thereof as measured in (a):
   (ba) with a respective healthy reference, whereby
      (baa) a comparable presence and/or quantity of the proBNP fragments and C-terminally truncated forms thereof in the sample to the presence and/or quantity in the healthy reference is indicative of absence of AHF, CHF or sepsis, and/or
      (bab) an altered presence and/or quantity of the proBNP fragments and C-terminally truncated forms thereof in the sample compared to the presence and/or quantity in the healthy reference is indicative of AHF, CHF or sepsis; and/or
   (bb) with a respective disease reference, whereby a comparable presence and/or quantity of the proBNP fragments and C-terminally truncated forms thereof in the sample to the presence and/or quantity in the disease reference is indicative of AHF, CHF or sepsis.

In an embodiment, step (a) of the above method may specifically measure two or more proBNP fragments selected from proBNP 3-108, 4-108 and 7-108 and C-terminally truncated forms thereof, and in step (b) a profile of presence and/or quantity of so-measured proBNP fragments is created and compared to a respective healthy reference profile and/or disease reference profile.

In another embodiment, step (a) of the above method may specifically measure two or more proBNP fragments selected from proBNP 1-108, 3-108, 4-108 and 7-108 and C-terminally truncated forms thereof, and in step (b) a profile of presence and/or quantity of so-measured proBNP fragments is created and compared to a respective healthy reference profile and/or disease reference profile.

In an embodiment, step (a) of the above method may specifically measure proBNP 4-108 and C-terminally truncated forms thereof, and the predicted or diagnosed condition is sepsis. The inventors have found that proBNP 4-108 fragment may be more typifying for sepsis.

In an embodiment, step (a) of the above method may specifically measure two or more proBNP fragments selected from proBNP 1-108, 3-108 and 4-108 and C-terminally truncated forms thereof, and in step (b) a profile of presence and/or quantity of so-measured proBNP fragments is created and compared to a respective healthy reference profile and/or disease reference profile; and the predicted or diagnosed condition is sepsis.

In an embodiment, step (a) of the above method may specifically measure proBNP 7-108 and C-terminally truncated forms thereof, and the predicted or diagnosed condition is AHF or CHF. The inventors have found that proBNP 7-108 fragment may be more typifying for AHF and CHF.

In an embodiment, step (a) of the above method may specifically measure two or more proBNP fragments selected from proBNP 1-108, 3-108 and 7-108 and C-terminally truncated forms thereof, and in step (b) a profile of presence and/or quantity of so-measured proBNP fragments is created and compared to a respective healthy reference profile and/or disease reference profile; and the predicted or diagnosed condition is AHF or CHF.

The above methods may optionally involve measuring further biomarkers relevant in AHF, CHF or sepsis, such as without limitation measuring other fragments of proBNP or NTproBNP, BNP and fragments thereof (see, for example, WO 2004/094460 for relevant fragments of BNP, such as inter alia BNP 3-32, BNP 1-29, BNP 1-30 and BNP 3-30), C-reactive protein (CRP) and/or procalcitonin (PCT).

So-measured additional biomarkers may be included in the comparison performed in step (b) of the methods.

Another aspect provides a method for the prediction, prognosis and/or diagnosis of AHF, CHF or sepsis in a subject comprising:
(a) assaying NTproBNP in a sample from the subject, comprising specifically measuring the presence and/or quantity in said sample of one or more fragments of NTproBNP selected from NTproBNP 3-76, 4-76 and 7-76 and C-terminally truncated forms thereof;
(b) comparing the presence and/or quantity of said one or more fragments of NTproBNP and C-terminally truncated forms thereof as measured in (a):
   (ba) with a respective healthy reference, whereby
      (baa) a comparable presence and/or quantity of the NTproBNP fragments and C-terminally truncated forms thereof in the sample to the presence and/or quantity in the healthy reference is indicative of absence of AHF, CHF or sepsis, and/or
      (bab) an altered presence and/or quantity of the NTproBNP fragments and C-terminally truncated forms thereof in the sample compared to the presence and/or quantity in the healthy reference is indicative of AHF, CHF or sepsis; and/or
   (bb) with a respective disease reference, whereby a comparable presence and/or quantity of the NTproBNP fragments and C-terminally truncated forms thereof in the sample to the presence and/or quantity in the disease reference is indicative of AHF, CHF or sepsis.

In an embodiment, step (a) of the above method may specifically measure two or more NTproBNP fragments selected from NTproBNP 3-76, 4-76 and 7-76 and C-terminally truncated forms thereof, and in step (b) a profile of presence and/or quantity of so-measured NTproBNP fragments is created and compared to a respective healthy reference profile and/or disease reference profile.

In another embodiment, step (a) of the above method may specifically measure two or more NTproBNP fragments selected from NTproBNP 1-76, 3-76, 4-76 and 7-76 and C-terminally truncated forms thereof, and in step (b) a profile of presence and/or quantity of so-measured

NTproBNP fragments is created and compared to a respective healthy reference profile and/or disease reference profile.

In an embodiment, step (a) of the above method may specifically measure NTproBNP 4-76 and C-terminally truncated forms thereof, and the predicted or diagnosed condition is sepsis. The inventors have found that NTproBNP 4-76 fragment may be more typifying for sepsis.

In an embodiment, step (a) of the above method may specifically measure two or more proBNP fragments selected from NTproBNP 1-76, 3-76 and 4-76 and C-terminally truncated forms thereof, and in step (b) a profile of presence and/or quantity of so-measured NTproBNP fragments is created and compared to a respective healthy reference profile and/or disease reference profile; and the predicted or diagnosed condition is sepsis.

In an embodiment, step (a) of the above method may specifically measure NTproBNP 7-76 and C-terminally truncated forms thereof, and the predicted or diagnosed condition is AHF or CHF. The inventors have found that NTproBNP 7-76 fragment may be more typifying for AHF and CHF.

In an embodiment, step (a) of the above method may specifically measure two or more NTproBNP fragments selected from NTproBNP 1-76, 3-76 and 7-76 and C-terminally truncated forms thereof, and in step (b) a profile of presence and/or quantity of so-measured NTproBNP fragments is created and compared to a respective healthy reference profile and/or disease reference profile; and the predicted or diagnosed condition is AHF or CHF.

The above methods may optionally involve measuring further biomarkers relevant in AHF, CHF or sepsis, such as without limitation measuring other fragments of proBNP or NTproBNP, measuring BNP and fragments thereof, CRP and/or PCT. So-measured additional biomarkers may be included in the comparison performed in step (b) of the methods.

As explained, some methods for assaying proBNP and NTproBNP may not discriminate at the C-terminal ends. Hence, the above aspects also include methods for the prediction, prognosis and/or diagnosis of AHF, CHF or sepsis in a subject, comprising:
(a) assaying proBNP and NTproBNP in a sample from the subject, comprising specifically measuring the presence and/or quantity in said sample of one or more fragments of proBNP and NTproBNP selected from proBNP 3-108 and NTproBNP 3-76, proBNP 4-108 and NTproBNP 4-76, or proBNP 7-108 and NTproBNP 7-76, and C-terminally truncated forms thereof;
(b) comparing the presence and/or quantity of said one or more fragments of proBNP and NTproBNP and C-terminally truncated forms thereof as measured in (a):
   (ba) with a respective healthy reference, whereby
      (baa) a comparable presence and/or quantity of the proBNP and NTproBNP fragments and C-terminally truncated forms thereof in the sample to the presence and/or quantity in the healthy reference is indicative of absence of AHF, CHF or sepsis, and/or
      (bab) an altered presence and/or quantity of the proBNP and NTproBNP fragments and C-terminally truncated forms thereof in the sample compared to the presence and/or quantity in the healthy reference is indicative of AHF, CHF or sepsis; and/or
   (bb) with a respective disease reference, whereby a comparable presence and/or quantity of the proBNP and NTproBNP fragments and C-terminally truncated forms thereof in the sample to the presence and/or quantity in the disease reference is indicative of AHF, CHF or sepsis.

In an embodiment, step (a) of the above method may specifically measure two or more proBNP and NTproBNP fragments selected from proBNP 3-108 and NTproBNP 3-76, proBNP 4-108 and NTproBNP 4-76, or proBNP 7-108 and NTproBNP 7-76, and C-terminally truncated forms thereof, and in step (b) a profile of presence and/or quantity of so-measured proBNP and NTproBNP fragments is created and compared to a respective healthy reference profile and/or disease reference profile.

In another embodiment, step (a) of the above method may specifically measure two or more proBNP and NTproBNP fragments selected from proBNP 1-108 and NTproBNP 1-76, proBNP 3-108 and NTproBNP 3-76, proBNP 4-108 and NTproBNP 4-76, or proBNP 7-108 and NTproBNP 7-76, and C-terminally truncated forms thereof, and in step (b) a profile of presence and/or quantity of so-measured proBNP fragments is created and compared to a respective healthy reference profile and/or disease reference profile.

In an embodiment, step (a) of the above method may specifically measure proBNP 4-108 and NTproBNP 4-76 and C-terminally truncated forms thereof, and the predicted or diagnosed condition is sepsis. The inventors have found that proBNP 4-108 and NTproBNP 4-76 fragments may be more typifying for sepsis.

In an embodiment, step (a) of the above method may specifically measure two or more proBNP and NTproBNP fragments selected from proBNP 1-108 and NTproBNP 1-76, proBNP 3-108 and NTproBNP 3-76, or proBNP 4-108 and NTproBNP 4-76, and C-terminally truncated forms thereof, and in step (b) a profile of presence and/or quantity of so-measured proBNP and NTproBNP fragments is created and compared to a respective healthy reference profile and/or disease reference profile; and the predicted or diagnosed condition is sepsis.

In an embodiment, step (a) of the above method may specifically measure proBNP 7-108 and NTproBNP 7-76 and C-terminally truncated forms thereof, and the predicted or diagnosed condition is AHF or CHF. The inventors have found that proBNP 7-108 and NTproBNP 7-76 fragments may be more typifying for AHF and CHF.

In an embodiment, step (a) of the above method may specifically measure two or more proBNP and NT proBNP fragments selected from proBNP 1-108 and NTproBNP 1-76, proBNP 3-108 and NTproBNP 3-76, or proBNP 7-108 and NTproBNP 7-76, and C-terminally truncated forms thereof, and in step (b) a profile of presence and/or quantity of so-measured proBNP and NTproBNP fragments is created and compared to a respective healthy reference profile and/or disease reference profile; and the predicted or diagnosed condition is AHF or CHF.

The above methods may optionally involve measuring further biomarkers relevant in AHF, CHF or sepsis, such as without limitation measuring other fragments of proBNP or NTproBNP, measuring BNP and fragments thereof, CRP and/or PCT. So-measured additional biomarkers may be included in the comparison performed in step (b) of the methods.

The invention also teaches establishing healthy references and diseases references for use in the above methods for predicting, prognosing and/or diagnosing AHF, CHF or sepsis.

Hence, an aspect provides a method for establishing a healthy reference or a disease reference for the presence and/or quantity of one or more fragments of proBNP, comprising:
(a) assaying proBNP in:
   (aa) a sample from one or more subjects not having acute heart failure (AHF), chronic heart failure (CHF) or sepsis, or
   (ab) a sample from one or more subjects having AHF, CHF or sepsis, comprising specifically measuring the presence and/or quantity in said sample (aa) or (ab) of one or more fragments of proBNP selected from proBNP 3-108, 4-108 and 7-108 and C-terminally truncated forms thereof;
(b) storing the presence and/or quantity of the respective fragments of proBNP and C-terminally truncated forms thereof
   (ba) as measured in (aa) as a healthy reference or a healthy reference profile, or
   (bb) as measured in (ab) as a disease reference or a disease reference profile.

Also provided is a method for establishing a healthy reference or a disease reference for the presence and/or quantity of one or more fragments of NTproBNP, comprising:
(a) assaying NTproBNP in:
   (aa) a sample from one or more subjects not having AHF, CHF or sepsis, or
   (ab) a sample from one or more subjects having AHF, CHF or sepsis, comprising specifically measuring the presence and/or quantity in said sample (aa) or (ab) of one or more fragments of NTproBNP selected from NTproBNP 3-76, 4-76 and 7-76 and C-terminally truncated forms thereof;
(b) storing the presence and/or quantity of the respective fragments of NTproBNP and C-terminally truncated forms thereof
   (ba) as measured in (aa) as a healthy reference or a healthy reference profile, or
   (bb) as measured in (ab) as a disease reference or a disease reference profile.

As explained, some methods for assaying proBNP and NTproBNP may not discriminate at the C-terminal ends. Hence, the above aspects also include methods for establishing a healthy reference or a disease reference for the presence and/or quantity of one or more fragments of proBNP and NTproBNP, comprising:
(a) assaying proBNP and NTproBNP in:
   (aa) a sample from one or more subjects not having AHF, CHF or sepsis, or
   (ab) a sample from one or more subjects having AHF, CHF or sepsis, comprising specifically measuring the presence and/or quantity in said sample (aa) or (ab) of one or more fragments of proBNP and NTproBNP selected from proBNP 3-108 and NTproBNP 3-76, proBNP 4-108 and NTproBNP 4-76, or proBNP 7-108 and NTproBNP 7-76, and C-terminally truncated forms thereof;
(b) storing the presence and/or quantity of the respective fragments of proBNP and NTproBNP and C-terminally truncated forms thereof
   (ba) as measured in (aa) as a healthy reference or a healthy reference profile, or
   (bb) as measured in (ab) as a disease reference or a disease reference profile.

The above reference profiles may optionally involve the presence and/or quantity of further biomarkers relevant in AHF, CHF or sepsis, such as without limitation other fragments of proBNP or NTproBNP, BNP and fragments thereof, CRP and/or PCT.

The isolated fragments of proBNP or NTproBNP and C-terminally truncated forms thereof as disclosed herein may be advantageously employed in the various assays and methods as taught herein, for example as positive controls, calibrators, etc.

Hence, a further aspect provides a protein array, e.g., a protein microarray, useful in assaying proBNP and/or NTproBNP, comprising one or more isolated fragments of proBNP and/or NTproBNP selected from proBNP 3-108, NTproBNP 3-76, proBNP 4-108, NTproBNP 4-76, proBNP 7-108, NTproBNP 7-76, or C-terminally truncated form of any one thereof, immobilised on a solid phase.

A yet further aspect provides a kit, useful in assaying proBNP and/or NTproBNP, comprising one or more isolated fragments of proBNP and/or NTproBNP selected from proBNP 3-108, NTproBNP 3-76, proBNP 4-108, NTproBNP 4-76, proBNP 7-108, NTproBNP 7-76, or C-terminally truncated form of any one thereof.

Further provided are isolated fragments of proBNP or NTproBNP selected from proBNP 3-108, NTproBNP 3-76, proBNP 4-108, NTproBNP 4-76, proBNP 7-108, NTproBNP 7-76, or C-terminally truncated form of any one thereof as taught herein, further comprising a detectable label. This facilitates ready detection of such fragments. Detection labels can be conventional depending on methods of detection, such as, without limitation, chromogenic, fluorogenic or radioactive moieties, peptide tags, haptens, isotopic or other mass labels, etc.

A further aspect provides specific-binding agents capable of specifically binding to any one or more of the fragments of proBNP or NTproBNP selected from proBNP 3-108, NTproBNP 3-76, proBNP 4-108, NTproBNP 4-76, proBNP 7-108, NTproBNP 7-76, or C-terminally truncated form of any one thereof as taught herein. Some specific-binding agents distinguish at least one of the above fragments from at least another of the above fragments.

In an embodiment, the specific-binding agent is capable of specifically binding to only one of the above fragments and C-terminally truncated forms thereof.

In a further embodiment, the specific-binding agent does not substantially bind to proBNP 1-108 and NTproBNP 1-76, and C-terminally truncated forms thereof. In an embodiment, the specific-binding agent may be chosen from the group comprising or consisting of an antibody, aptamer, photoaptamer, protein, peptide, peptidomimetic or a small molecule.

In a further embodiment the specific-binding agent comprises a detectable label.

Further provided is a method for immunising an animal, preferably a warm-blooded animal, more preferably a mammal, even more preferably a non-human animal or mammal, using any of the novel proBNP and/or NTproBNP fragments and/or C-terminally truncated forms thereof as disclosed herein, optionally fused to or otherwise covalently or non-covalently linked, bound or adsorbed to a presenting carrier.

A further aspect covers immune sera and antibody reagents, particularly antibody reagents directed against the immunising proBNP and/or NTproBNP fragments and/or C-terminally truncated forms thereof, isolated from so-immunised animals.

Also provided is a method of selecting the specific-binding agent as taught herein comprising:
(a) providing a plurality of test specific-binding agents;
(b) selecting from the test specific-binding agents of (a) specific-binding agents which bind to one or more desired isolated fragments of proBNP or NTproBNP and C-terminally truncated forms thereof as taught herein,
(c) removing from the specific-binding agents selected in (b) those which also bind to any of the not desired isolated fragments of proBNP or NTproBNP and C-terminally truncated forms thereof as taught herein and/or to proBNP 1-108 or NTproBNP 1-76 and C-terminally truncated forms thereof.

Further provided is use of the specific-binding agents in any of the assays and methods disclosed herein, and assays such as immunoassays using said specific-binding agents.

Also provided is a kit, useful in assaying proBNP and/or NTproBNP, comprising one or more specific-binding agents as taught herein.

In any of the methods of the invention described herein, the measurement may preferably be specific for the NTproBNP 3-76, 4-76 or proBNP 3-108 or 4-108 fragment, or C-terminally truncated fragments thereof and/or the measurement is preferably specific for NTproBNP or proBNP but insensitive for the proBNP 7-108 or NTproBNP 7-76 fragment or N- and/or C-terminally truncated fragments thereof.

In any of the kits, assays or methods of the invention described herein, the binding agent may preferably be specific for the NTproBNP 3-76, 4-76 or proBNP 3-108 or 4-108 fragment, or C-terminally truncated fragments thereof and/or preferably specific for NTproBNP or proBNP but insensitive for the proBNP 7-108 or NTproBNP 7-76 fragment or N- and/or C-terminally truncated fragments thereof.

The inventors further established that specifically measuring the level of said different proBNP and/or NTproBNP fragments selected from proBNP 3-108, NTproBNP 3-76, proBNP 4-108, NTproBNP 4-76, proBNP 7-108, NTproBNP 7-76, or C-terminally truncated form of any one thereof as taught herein, greatly improves the reliability of the prognosis and/or diagnosis and/or prediction of disorders or diseases for which measuring of BNP-protein processing is relevant such as acute heart failure (AHF), chronic heart failure (CHF) or sepsis.

The inventors have shown that the change in protein level of the proBNP 3-108, NTproBNP 3-76, proBNP 4-108, and NTproBNP 4-76 fragments generally follows the same trend when measured in patients having AHF or sepsis, namely their level decreases with improved prognostics or their level is higher in patients having a high risk of acute or chronic heart failure or sepsis as compared to low risk patients.

The change in protein levels of proBNP 7-108, NTproBNP 7-76 fragments on the other hand follows an unpredictable trend, when analysing patients upon admission to the hospital with AHF or sepsis and upon dismissal (i.e. the level goes up upon dismissal of some patients, and goes down upon dismissal of other patients). This unpredictable pattern correlates with the unpredictable pattern of BNP measured by commonly used ELISA in the hospital, resulting in the large grey-zone as mentioned in the introduction.

The invention thus now provides for the first time evidence for the cause of this large grey zone in BNP protein level and uses this knowledge to provide new tools for assessing the risk of AHF, CHF or sepsis in a subject in a more accurate and reliable and trustworthy manner.

On the one hand, the invention provides for methods of diagnosis, prognosis or predicting the occurrence of AHF or sepsis, wherein the detection of the proBNP 7-108, NTproBNP 7-76 fragments or C-terminally truncated form of any one thereof as taught herein is avoided.

On the other hand, the invention provides for methods of diagnosis, prognosis or predicting the occurrence of AHF or sepsis, wherein the detection of the proBNP 3-108, NTproBNP 3-76, proBNP 4-108, NTproBNP 4-76 fragments or C-terminally truncated form of any one thereof as taught herein is specifically envisaged through either Mass-Spectroscopic means or ELISA using specifically designed binding molecules or other specific detection methods.

Preferably, the detection methods are specific for the NTproBNP 3-76 and/or 4-76 or proBNP 3-108 and/or 4-108 fragments and C-terminally truncated forms thereof or are insensitive for the NTproBNP 7-76 and/or proBNP 7-108 fragment and further N-terminally and C-terminally truncated fragments thereof. Without wanting to be bound to any theory, the inventors believe that truncated forms of NTproBNP or proBNP that lack more than 6 amino acids N-terminally, are less relevant in diagnosis and might even perturb the diagnostic value of (NT)proBNP.

The invention therefore provides for a method for predicting, diagnosing or prognosing recovery of sepsis or heart failure in a subject, comprising; a) the measurement of one or more of the NTproBNP fragments 3-76 or 4-76, or BNP fragments 3-108 and 4-108 or C-terminally truncated forms thereof, at a first time point; b) the measurement of one or more of the NTproBNP fragments 3-76 or 4-76, or BNP fragments 3-108 and 4-108 or C-terminally truncated forms thereof, at a later time point; and c) comparing both values, wherein a decrease in amount of either of the one or more fragments at the later time point as compared to the level at the first time point reflects recovery of heart failure of the patient. Optionally, said method additionally comprises the steps of: d) measuring the total amount of BNP or alternatively measuring the amount of NTproBNP 7-76 or BNP 7-108 or further N-terminally and C-terminally truncated forms thereof; and e) calculating the ratio of the values obtained in steps a-b versus the value obtained in step d) in order to compensate for measurement errors. In a preferred embodiment of said method, both fragments of NTproBNP 3-76 and 4-76 or BNP fragments 3-108 and 4-108 or C-terminally truncated forms thereof, are measured simultaneously.

In an alternative embodiment, the invention provides an assay for predicting, diagnosing or prognosing recovery of sepsis or heart failure in a subject, comprising; a) tools for specific measurement of one or more of the NTproBNP fragments 3-76 or 4-76, or BNP fragments 3-108 or 4-108 or C-terminally truncated forms thereof, in a sample of a patient; b) instructions for measuring one or more of the NTproBNP fragments 3-76 or 4-76, or BNP fragments 3-108 and 4-108 or C-terminally truncated forms thereof, at a first time point and at a second time point after the first time point and for comparing both values, wherein a decrease in amount of either of the one or more fragments at the second time point as compared to the level at the first time point indicates a recovery of heart failure. Preferably, the above method additionally comprises tools to measure the total amount of BNP or the amount of NTproBNP 7-76 or BNP 7-108 or further N-terminally and C-terminally truncated forms thereof and instructions to calculate the ratio of the levels of a) the NTproBNP fragments 3-76 or 4-76, or BNP fragments 3-108 and 4-108 or C-terminally truncated forms thereof versus b) the total amount of BNP or the amount of NTproBNP 7-76 or BNP 7-108 or further N-terminally and C-terminally truncated forms thereof. In certain preferred embodiments, both fragments of NTproBNP 3-76 and 4-76 or BNP fragments 3-108 and 4-108 or C-terminally truncated forms thereof are measured simultaneously. The invention also provides for the use of said assay in predicting, diagnosing or prognosing recovery of heart failure in a subject.

The invention further provides a method for prognosis, diagnosis and or prediction of sepsis or heart failure in a subject comprising; a) the measurement of one or more of the NTproBNP fragments 3-76 or 4-76, or BNP fragments 3-108 and 4-108 or C-terminally truncated forms thereof; b) comparing the amount of the measurement in step a) to the level of said fragment(s) in a healthy subject, wherein an increase in amount of either of the one or more fragments in the patient as compared to the healthy subject points towards a high risk of sepsis or heart failure. In a preferred embodiment, said method additionally measures the total amount of BNP or the amount of NTproBNP 7-76 or BNP 7-108 or further N-terminally and C-terminally truncated forms thereof and the ratio of a) the NTproBNP fragments 3-76 or 4-76, or BNP fragments 3-108 and 4-108 or C-terminally truncated forms thereof versus b) the total amount of BNP or the amount of NTproBNP 7-76 or BNP 7-108 further N-terminally and or C-terminally truncated forms thereof, is calculated in order to compensate for measurement errors. In a further preferred embodiment, both fragments of NTproBNP 3-76 and 4-76 or BNP fragments 3-108 and 4-108 or C-terminally truncated forms thereof are measured simultaneously.

Additionally, the invention provides a method for prognosis, diagnosis and or prediction of sepsis or heart failure in a hospitalised subject comprising: a) the measurement of one or more of the NTproBNP fragments 3-76 or 4-76, or BNP fragments 3-108 and 4-108 or C-terminally truncated forms thereof at a first time point; b) the measurement of one or more of the NTproBNP fragments 3-76 or 4-76, or BNP fragments 3-108 and 4-108 or C-terminally truncated forms thereof at a later time point; c) comparing the amounts of the measurement(s) in step a) to the measurement(s) in step b), wherein an increase in amount of either of the one or more fragments in step b) compared to the level in step a) points towards a high risk of heart failure. A decrease of the amount of either of the one or more fragments in step b) compared to the level in step a) points towards a lower risk of heart failure (i.e. recovery), while no change in the amount of either of the one or more fragments in step b) compared to the level in step a) points towards lack of change of the disease condition in the tested patient.

Alternatively, the invention provides a method for the prognosis, diagnosis and or prediction of heart failure in a subject comprising: a) the measurement of the amount of NTproBNP in a sample from a subject, wherein said amount of NTproBNP does not include the 7-76 or 7-108 fragment or further N-terminally and C-terminally truncated forms thereof; and b) comparing said amount of NTproBNP measured in step a) with the level of NTproBNP not including the 7-76 or 7-108 fragment or further N-terminally and C-terminally truncated forms thereof in a healthy subject, wherein an increase in the amount of NTproBNP points towards an increased risk of heart failure. A decrease of the amount of either of the one or more fragments in step b) compared to the level in step a) points towards a lower risk of heart failure (i.e. recovery), while no change in the amount of either of the one or more fragments in step b) compared to the level in step a) points towards lack of change of the disease condition in the tested patient.

Furthermore, the invention provides a method for assessing the risk of suffering from heart failure in a subject comprising: a) measuring the total amount of NTproBNP in a sample; and b) measuring specifically the fragment 7-76 of NTproBNP or 7-108 of BNP or further N-terminally and C-terminally truncated forms thereof; c) calculating the total amount of NTproBNP in the sample minus the amount of the fragment 7-76 of NTproBNP or 7-108 of BNP or further N-terminally and C-terminally truncated forms thereof; and d) establish the difference between the measurement in step c) from the patient and the measurement in step c) from a healthy subject, wherein an increase of said measurement in step c) in the patient as compared to a healthy subject indicates a high risk of suffering from heart failure and a decrease in said measurement in step c) in the patient as compared to a healthy subject indicates a low risk of suffering from heart failure. No change in the amount of either of the one or more fragments in step b) compared to the level in step a) points towards lack of change of the disease condition in the tested patient.

In addition, the invention provides an assay for assessing the risk of suffering from heart failure in a subject comprising means for specifically measuring the level of NTproBNP in a sample from a subject, wherein said means is specifically insensitive to the NTproBNP fragment 7-76 or further N-terminally and C-terminal truncated forms thereof. The invention also provides the use of said assay in assessing the risk of suffering from heart failure in a subject.

In a further embodiment, the invention provides a method for assessing the risk of suffering from heart failure in a subject comprising specifically measuring the level of NTproBNP in a sample from a subject, wherein the means used for measuring NTproBNP is specifically insensitive to the NTproBNP fragment 7-76 or further N-terminally and C-terminal truncated forms thereof.

The invention also provides means for detecting NTproBNP in a sample, the means being selected from one or more antibodies, aptamers, photoaptamers, proteins, peptides, peptidomimetics or a small molecules, characterised in that said means is specifically insensitive to the NTproBNP fragment 7-76 or further N-terminally and C-terminal truncated forms thereof. In an alternative embodiment, the invention provides the use of said means in assessing the risk of suffering from heart failure in a subject.

In one preferred embodiment of the methods and/or assays of the invention as described herein, the quantification of said specific proBNP or NTproBNP fragment selected from the group of proBNP fragments 3-108, 4-108 and 7-108, or a C-terminally truncated form of any one thereof and/or the NTproBNP fragments 3-76, 4-76 and 7-76 or further N-terminally truncated forms of the proBNP 7-108 or NTproBNP 7-76 fragments, or C-terminally truncated forms of any one thereof in a sample of a subject is done by Mass-spectrometry, comprising the steps of:

a) adding to the sample a known amount of any one of the reference peptides of any one of claims 1-7, that are labelled or mass-altered;
b) performing Mass-Spectrometric analysis of the sample;
c) determining the surface of the peak (1) corresponding to the reference peptide;
d) determining the surface of the peak (2) corresponding to the target peptide;
e) calculating the ratio of peaks 1 and 2; and
f) calculating the exact amount of the target protein, based on the ratio of step e) and the known amount of reference peptide added to the sample in step a).

The invention thus further provides for a kit for use in said methods and/or assays of the invention, comprising one or more isolated fragments of proBNP or NTproBNP or C-terminally truncated form thereof according to the invention and described herein.

The invention thus further provides for a kit for use in said methods and/or assays of the invention, comprising one or more specific-binding agents according to the invention and described herein.

These and further aspects and preferred embodiments of the invention are described in the following sections and in the appended claims.

### BRIEF DESCRIPTION OF FIGURES

FIG. 1 illustrates sequences of preproBNP and peptides derived there from.

FIG. 2 presents some exemplary experimental data as obtained from example 1, experiment 2 (vide infra): i.e., the extraction ion chromatograms corresponding the m/z-values of the ¹⁸O-labelled triply charged [M+3H⁺]³⁺ sequences of N-terminal acetylated-HPLGSPGSASDLETSGLQEQER (SEQ ID NO: 5) and N-terminal acetylated-LGSPGSASDLETSGLQEQER (SEQ ID NO: 6); the raw MS/MS spectra leading to the positive identification of the latter sequences as well as the ¹⁶O/¹⁸O isotopic envelopes observed for the differentially labelled acetylated-HPLGSPGSASDLETSGLQEQER, acetylated-LGSPGSASDLETSGLQEQER and a random peptide demonstrating no differential behaviour.

FIG. 3 shows three different measurement methods of BNP: Top panel shows the standard Cofradic™ method revealing one BNP isoform, measured in 5 of 8 plasma samples of acute heart failure patients. The middle panel shows an improved SCX (strong cation exchange) column-based mass-spectrometry method developed by the inventors, revealing three different isoforms of BNP, measured in each of the 8 plasma samples of acute heart failure patients; The lower panel shows a common used ELISA detection method for BNP, not enabling distinction between any of the proBNP or NT-proBNP isoforms identified by the present invention.

FIG. 4 shows the analysis of paired samples of patients, wherein each pair of samples is derived from the same patient, but at two different time points, upon admission to the hospital and upon dismissal of the patient form the hospital after being treated for AHF. As can be seen from the figure, three NTproBNP fragments, the NTproBNP 3-76, NTproBNP 4-76 and NTproBNP 7-76 are detectable in the samples. Overall the different fragments show the same behaviour in these 3 patients from admission to discharge.

FIG. 5 shows the comparison of our MS-based read-out for NTproBNP 3-76 with the ELISA based measurements for BNP and NTproBNP in 3 different patients. While in patients Phu12 and Phu28 there is good correlation between ELISA and NTproBNP 3-76, in patient Phu38 we observe an opposite trend for BNP and NTproBNP 3-76.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the terms “pro-B-type natriuretic peptide” (also abbreviated as “proBNP”) and “amino terminal pro-B-type natriuretic peptide” (also abbreviated as “NTproBNP”) and B-type natriuretic peptide (also abbreviated as “BNP”) refer to peptides commonly known under these designations in the art. As further explanation and without limitation, in vivo proBNP, NTproBNP and BNP derive from natriuretic peptide precursor B preproprotein (preproBNP). In particular, proBNP peptide corresponds to the portion of preproBNP after removal of the N-terminal secretion signal (leader) sequence from preproBNP. NTproBNP corresponds to the N-terminal portion and BNP corresponds to the C-terminal portion of the proBNP peptide subsequent to cleavage of the latter C-terminally adjacent to amino acid 76 of proBNP.

The terms encompass such peptides from any organism where found, and particularly from animals, preferably vertebrates, more preferably mammals, including humans and non-human mammals, even more preferably from humans.

The designations proBNP, NTproBNP and BNP as used herein particularly refer to such peptides with a native sequence, i.e., peptides of which the primary sequence is the same as that of respectively proBNP, NTproBNP or BNP found in or derived from nature. A skilled person understands that native sequences of proBNP, NTproBNP or BNP may differ between different species due to genetic divergence between such species. Moreover, the native sequences of proBNP, NTproBNP or BNP may differ between or even within different individuals of the same species due to normal genetic diversity (variation) within a given species. Also, the native sequences of proBNP, NTproBNP or BNP may differ between or even within different individuals of the same species due to post-transcriptional or post-translational modifications. Accordingly, all proBNP, NTproBNP or BNP sequences found in or derived from nature are considered “native”.

The designations proBNP, NTproBNP or BNP as used herein encompass the respective peptides when forming a part of a living organism, organ, tissue or cell, when forming a part of a biological sample, as well as when at least partly isolated from such sources. The terms also encompass the respective peptides when produced by recombinant or synthetic means.

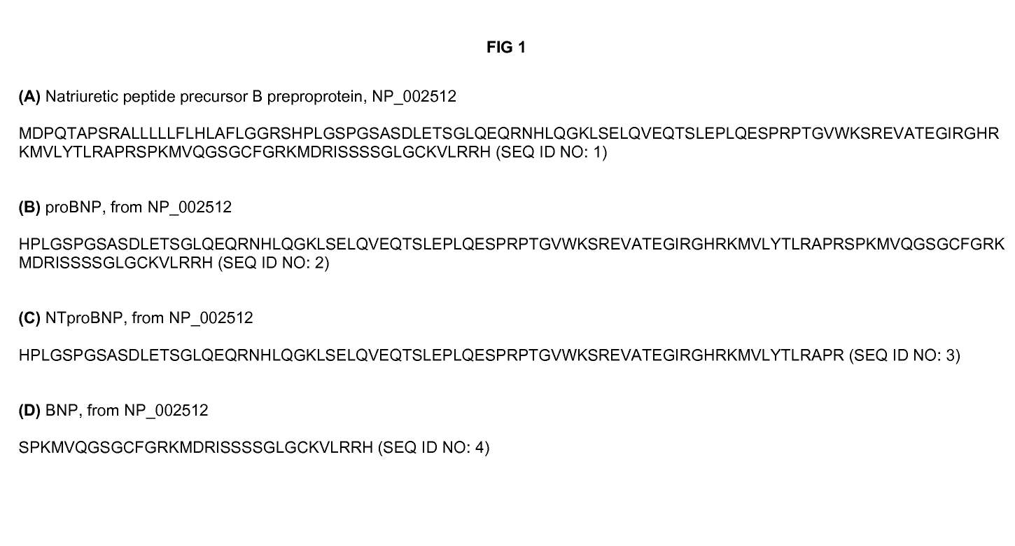

Exemplary human proBNP peptide includes without limitation the peptide from amino acid position 27 to position 134 of the natriuretic peptide precursor B preproprotein sequence as annotated under the NIH Entrez Protein (http://www.ncbi.nlm.nih.gov/sites/entrez?db=protein) accession number NP_{—}002512 (version NP_{—}002512.1 revised Apr. 20, 2008). The sequence of NP_{—}002512 is shown in FIG. 1A (SEQ ID NO: 1) and the exemplary sequence of proBNP from NP_{—}002512 is shown in FIG. 1B (SEQ ID NO: 2). Exemplary human NTproBNP peptide includes without limitation the peptide from amino acid position 27 to position 102 of the natriuretic peptide precursor B preproprotein sequence as annotated under said NIH Entrez Protein accession number NP_{—}002512. The exemplary sequence of NTproBNP from NP_{—}002512 is shown in FIG. 1C (SEQ ID NO: 3). Exemplary human BNP peptide includes without limitation the peptide from amino acid position 103 to position 134 of the natriuretic peptide precursor B preproprotein sequence as annotated under said NIH Entrez Protein accession number NP_{—}002512. The exemplary sequence of BNP from NP_{—}002512 is shown in FIG. 1D (SEQ ID NO: 4). See also Sudoh et al. 1989 (Biochem Biophys Res Commun 159: 1427-1434) for further exemplification of human preproBNP-derived peptides, including proBNP, NTproBNP and BNP.

The term “fragment” of proBNP, NTproBNP or BNP generally refers to a peptide that has an N-terminal and/or C-terminal deletion of one or more amino acid residues as compared to proBNP, NTproBNP or BNP, but where the remaining primary sequence of the fragment is identical to the corresponding positions in the amino acid sequence of proBNP, NTproBNP or BNP.

The terms proBNP 3-108, proBNP 4-108 or proBNP 7-108 denote fragments of proBNP, wherein the primary sequence of said fragments corresponds respectively to positions 3-108, 4-108 or 7-108 of a proBNP peptide, such as for example the human proBNP peptide shown in FIG. 1B.

The terms NTproBNP 3-76, NTproBNP 4-76 or NTproBNP 7-76 denote fragments of NTproBNP, wherein the primary sequence of said fragments corresponds respectively to positions 3-76, 4-76 or 7-76 of a NTproBNP peptide, such as for example the human NTproBNP peptide shown in FIG. 1C.

The term “C-terminally truncated form” with reference to a peptide, polypeptide or fragment thereof generally denotes such form that has a C-terminal deletion of one or more amino acid residues as compared to said peptide, polypeptide or fragment. Preferably, a C-terminally truncated form of a peptide, polypeptide or fragment thereof may retain 6, 7, 8, 9, such as 10 or 15, or even such as 20, 30, 50 or even such as 100 contiguous amino acids starting from the N-terminus of said peptide, polypeptide or fragment (symbol “≧” is synonymous with expressions “at least” or “equal to or more”).

Unless otherwise apparent from the context, reference herein to preproBNP, proBNP, NTproBNP, BNP, the herein disclosed fragments and any C-terminally truncated forms thereof also encompasses modified forms of such peptides bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “isolated” with reference to a particular component (such as for instance, a peptide, polypeptide or fragment thereof) generally denotes that such component exists in separation from—for example, has been separated from or prepared in separation from—one or more other components of its natural environment. For instance, an isolated human or animal peptide, polypeptide or fragment exists in separation from a human or animal body where it occurs naturally.

The term “isolated” as used herein may preferably also encompass the qualifier “purified”. As used herein, the term “purified” in reference to peptide(s), polypeptide(s) and/or fragment(s) thereof does not require absolute purity. Instead, it denotes that such peptide(s), polypeptide(s) and/or fragment(s) is (are) in a discrete environment in which their abundance (conveniently expressed in terms of mass or weight) relative to other proteins is greater than in a biological sample. A discrete environment denotes a single medium, such as for example a single solution, gel, precipitate, lyophilisate, etc. Purified peptides, polypeptides or fragments may be obtained by known methods including, for example, laboratory or recombinant synthesis, chromatography, preparative electrophoresis, centrifugation, precipitation, affinity purification, etc.

Purified peptide(s), polypeptide(s) and/or fragment(s) may preferably constitute by weight ≧10%, more preferably ≧50%, such as ≧60%, yet more preferably ≧70%, such as ≧80%, and still more preferably ≧90%, such as ≧95%, ≧96%, ≧97%, ≧98%, ≧99% or even ≧100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of peptides or polypeptides may be determined by SDS-PAGE under reducing or non-reducing conditions using Coomassie blue or, preferably, silver stain.

Where the herein described proBNP, NTproBNP, BNP or fragments and C-terminally truncated forms thereof are said to be “human”, this denotes that their primary sequence is the same as a corresponding primary sequence of or present in a naturally occurring human proBNP, NTproBNP, BNP or fragments thereof. Hence, the qualifier “human” in this connection relates to the primary sequence of the respective peptides, rather than to their origin or source. For example, such peptides may be present in or isolated from samples of human subjects or may be obtained by other means (e.g., by recombinant expression, cell-free translation or non-biological peptide synthesis).

In an embodiment, C-terminally truncated forms of the disclosed proBNP or NTproBNP fragments may be obtained or obtainable by proteolysis of said fragments to achieve advantageously detectable N-terminal forms.

For example, such proteolysis may be performed by suitable physical, chemical and/or enzymatic agents, more preferably chemical and/or enzymatic agents, even more preferably enzymatic agents, e.g., proteinases, preferably endoproteinases. Preferably, the proteolysis may be achieved by one or more, preferably one, endoproteinase, i.e., a protease cleaving internally within a protein or polypeptide chain. A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases.

Exemplary non-limiting endoproteinases include trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). The invention encompasses the use of any further known or yet to be identified enzymes; a skilled person can choose suitable protease(s) on the basis of their cleavage specificity and frequency to achieve desired protein peptide mixtures.

Preferably, the proteolysis may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, trypsin in solution, trypsin immobilised to a solid support, etc. Trypsin is particularly useful, inter alia due to high specificity (C-terminally adjacent to Arg and Lys except where the next residue is Pro) and efficiency of cleavage. The invention also contemplates the use of any trypsin-like protease, i.e., with a similar specificity to that of trypsin.

In other embodiments, chemical reagents may be used for proteolysis. For example, CNBr can cleave at Met; BNPS-skatole can cleave at Trp.

The conditions for treatment, e.g., protein concentration, enzyme or chemical reagent concentration, pH, buffer, temperature, time, can be determined by the skilled person depending on the enzyme or chemical reagent employed.

The terms proBNP or NTproBNP 3-21, 4-21 or 7-21 denote fragments of proBNP or NTproBNP, wherein the primary sequence of said fragments corresponds respectively to positions 3-21, 4-21 or 7-21 of a proBNP or NTproBNP peptide, such as for example the human proBNP or NTproBNP peptides shown in FIGS. 1B and C, respectively.

The present novel fragments of proBNP or NTproBNP and C-terminally truncated forms thereof are useful biomarkers.

The term “biomarker” as used herein generally refers to a biological molecule which is differentially present in samples from subjects having a genotype or phenotype of interest and/or who have been exposed to a condition of interest (query sample), as compared to equivalent samples from control subjects not having said genotype or phenotype and/or not having been exposed to said condition (control sample). The phrase “differentially present” refers to a demonstrable, preferably statistically significant, difference in the presence or quantity of the said biological molecule between one or a set of query samples compared to one or a set of control samples. Suitable biomarkers may include without limitation a protein or fragment thereof, a peptide, a polypeptide, a proteoglycan, a glycoprotein, a lipoprotein, a carbohydrate, a lipid, a nucleic acid, or other polymer, or any biological molecule that is present in a biological sample and that may be isolated from, or measured in, the biological sample. Furthermore, a biomarker can be an entire biological molecule or a part thereof that may be at least partly functional or recognised, for example, by an antibody, aptamer or other specific binding molecule. A biomarker may also be constituted by a certain aspect or form of a given biological molecule, such as for example a mutein or a modified form (phosphorylation, sulphonation, glycosylation, cleavage, etc.) thereof.

It shall be appreciated that methods for prediction, prognosis and/or diagnosis of various conditions may generally include measuring the presence (e.g., readout being present vs. absent; or detectable amount vs. undetectable amount) or quantity (e.g., readout being absolute quantity, relative quantity or concentration) of one biomarker or of two or more distinguishable biomarkers (also encompassing measuring two or more aspects of the same biological molecule) in biological samples from subjects.

When two or more different biomarkers are determined in a subject, their respective presence and/or quantity may be together represented as a biomarker profile, the values for each measured biomarker making a part of said profile. As used herein, the term “profile” includes any set of data that represents the distinctive features or characteristics associated with a condition of interest, such as particularly AHF, CHF or sepsis. The term encompasses inter alia nucleic acid profiles, such as for example genotypic profiles (sets of genotypic data that represents the genotype of one or more genes associated with a condition of interest), gene copy number profiles (sets of gene copy number data that represents the amplification or deletion of one or more genes associated with a condition of interest), gene expression profiles (sets of gene expression data that represents the mRNA levels of one or more genes associated with a condition of interest), DNA methylation profiles (sets of methylation data that represents the DNA methylation levels of one or more genes associated with a condition of interest), as well as protein profiles, such as for example protein expression profiles (sets of protein expression data that represents the levels of one or more proteins associated with a condition of interest), protein activation profiles (sets of data that represents the activation or inactivation of one or more proteins associated with a condition of interest), protein modification profiles (sets of data that represents the modification of one or more proteins associated with a condition of interest), protein cleavage profiles (sets of data that represent the proteolytic cleavage of one or more proteins associated with a condition of interest), as well as any combinations thereof.

Biomarker profiles may be created in a number of ways and may be a ratio of two or more measurable biomarkers or aspects of biomarkers. A biomarker profile comprises at least two measurements, where the measurements can correspond to the same or different biomarkers. A biomarker profile may also comprise at least three, four, five, 10, 20, 30 or more measurements. In one embodiment, a biomarker profile comprises hundreds, or even thousands, of measurements.

The invention also provides methods for assaying proBNP and/or NTproBNP in samples, comprising specifically measuring the presence and/or quantity of one or more of the novel proBNP and/or NTproBNP fragments and C-terminally truncated forms as taught herein.

The term “sample” as used herein includes any biological specimen obtained from a subject. Samples include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. In preferred embodiments, the sample is whole blood or a fractional component thereof such as plasma, serum, or a cell pellet. Preferably the sample is readily obtainable by minimally invasive methods. Samples may also include tissue samples and biopsies, tissue homogenates and the like.

A molecule or analyte such as a peptide or polypeptide, or a group of two or more molecules or analytes such as two or more peptides or polypeptides, is “specifically measured” when the presence and/or quantity of said molecule or said group of molecules is detected in a sample substantially to the exclusion of other molecules that are structurally related. For example, one proBNP polypeptide selected from the group consisting of proBNP 3-108, 4-108 and 7-108 and C-terminally truncated forms thereof is specifically measured when the measurement detects that polypeptide in a manner distinguishable from measurement of any other proBNP polypeptide in said group, and distinguishable from any measurement of proBNP 1-108 and C-terminally truncated forms thereof. For example, one NTproBNP polypeptide selected from the group consisting of NTproBNP 3-76, 4-76 and 7-76 and C-terminally truncated forms thereof is specifically measured when the measurement detects that polypeptide in a manner distinguishable from measurement of any other NTproBNP polypeptide in said group, and distinguishable from any measurement of NTproBNP 1-76 and C-terminally truncated forms thereof. For example, one combination or pair of proBNP and NTproBNP polypeptides selected from the group consisting of proBNP 3-108 and NTproBNP 3-76, proBNP 4-108 and NTproBNP 4-76, or proBNP 7-108 and NTproBNP 7-76, and C-terminally truncated forms thereof, is specifically measured when the measurement detects that pair of proBNP and NTproBNP polypeptides in a manner distinguishable from measurement of any other proBNP and NTproBNP polypeptides in said group, and distinguishable from any measurement of proBNP 1-108, NTproBNP 1-76 and C-terminally truncated forms thereof. Preferably, the specific measurement detects NTproBNP 3-76 and/or 4-76 or proBNP 3-108 and/or 4-108 fragments and C-terminally truncated forms thereof or is insensitive for the NTproBNP 7-76 and/or proBNP 7-108 fragment and further N-terminally and C-terminally truncated fragments thereof. Without wanting to be bound to any theory, the inventors have reasons to believe that that truncated forms of NTproBNP or proBNP that lack more than 6 amino acids N-terminally, are less relevant in diagnosis and might even perturb the diagnostic value of (NT)proBNP. These N-terminally truncated fragments may be lacking normal (NT)proBNP activity and therefore do not necessarily correlate with the physiological influence of BNP on the disease state. Since BNP activity has been shown to be relevant for determining certain disease states such as AHF, CHF and sepsis, an accurate test of its activity is needed; By excluding the N-terminally truncated forms of NTproBNP or proBNP that lack more than 6 amino acids N-terminally, the invention provides a more accurate tool for measuring BNP activity rather than measuring partial or total BNP protein or peptide fragment content in the sample.

Any available or conventional separation, detection and quantification methods can be used in the present invention to specifically measure the presence and/or quantity of the one or more novel proBNP and/or NTproBNP fragments and C-terminally truncated forms as disclosed herein, and optionally to measure other preproBNP derived molecules and other biomarkers of interest (any molecules of interest to be so-measured in a sample may be herein below referred to as biomarkers). For example, such methods may include immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly an antibody, and the molecule(s) or analyte(s) of interest.

The term “specifically bind” as used throughout this specification means that an agent (referred to herein as “specific-binding agent”) binds to one or more desired molecules or analytes, such as to one or more peptides or polypeptides of interest or fragments thereof, substantially to the exclusion of other molecules that are structurally related, as well as substantially to the exclusion of other molecules which are random or unrelated.

The term “specifically bind” does not necessarily require that an agent binds exclusively to its intended target(s). For example, an agent may be said to specifically bind to peptide(s), polypeptide(s) and/or fragment(s) thereof of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, a specific-binding agent may bind to its intended target(s) with affinity constant (K_{A}) of such binding K_{A}≧1×10⁶ M⁻¹, more preferably K_{A} ≧1×10⁷ M⁻¹, yet more preferably K_{A} ≧1×10⁸ M⁻¹, even more preferably K_{A}≧1×10⁹ M⁻¹, and still more preferably K_{A}≧1×10¹⁹ M⁻¹ or K_{A}≧1×10¹¹ M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

In an embodiment, a specific-binding agent as intended herein may be an antibody. Antibodies are particularly suited as specific-binding agents used in immunoassays.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

In an embodiment, an antibody may be any of IgA, IgD, IgE, IgG and IgM classes, and preferably IgG class antibody.

In an embodiment, the antibody may be a polyclonal antibody, e.g., an antiserum or immunoglobulins purified there from (e.g., affinity-purified).

In another preferred embodiment, the antibody may be a monoclonal antibody or a mixture of monoclonal antibodies. Monoclonal antibodies can target a particular antigen or a particular epitope within an antigen (such as required for targeting the novel proBNP and NTproBNP fragments) with greater selectivity and reproducibility.

By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

In further embodiments, the antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Immunoassay technologies include without limitation direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary antibody to bind to and thereby quantify target antigen in a sample immobilised on a solid support such as a microwell plate. Indirect ELISA uses a non-labelled primary antibody which binds to the target antigen and a secondary labelled antibody that recognises and allows to quantify the antigen-bound primary antibody. In sandwich ELISA the target antigen is captured from a sample using an immobilised ‘capture’ antibody which binds to one antigenic site within the antigen, and subsequent to removal of non-bound analytes the so-captured antigen is detected using a ‘detection’ antibody which binds to another antigenic site within said antigen, where the detection antibody may be directly labelled or indirectly detectable as above. Competitive ELISA uses a labelled ‘competitor’ that may either be the primary antibody or the target antigen. In an example, non-labelled immobilised primary antibody is incubated with a sample, this reaction is allowed to reach equilibrium, and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the end-point is typically colorimetric, chemiluminescent or fluorescent.

Radioimmunoassay (RIA) is a competition-based technique and involves mixing known quantities of radioactively-labelled (e.g., ¹²⁵I- or ¹³¹I-labelled) target antigen with antibody to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Further, mass spectrometry methods are suitable for measuring the herein disclosed novel proBNP and NTproBNP fragments and C-terminally truncated forms thereof, and any other biomarkers of relevance for the present disclosure.

Generally, any mass spectrometric (MS) techniques that can obtain precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein.

MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID).

In an embodiment, detection and quantification of biomarkers by mass spectrometry may involve multiple reaction monitoring (MRM), such as described among others by Kuhn et al. 2004 (Proteomics 4: 1175-86).

In an embodiment, MS peptide analysis methods may be advantageously combined with upstream peptide or protein separation or fractionation methods, such as for example with the chromatographic and other methods described herein below.

Chromatography can also be used for measuring the herein disclosed novel proBNP and NTproBNP fragments and C-terminally truncated forms thereof, and any other biomarkers of relevance for the present disclosure. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993.

Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

In an embodiment, chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The invention provides methods for the prediction, prognosis and/or diagnosis of certain diseases or conditions or predispositions thereto in subjects. The term “subject” or “patient” as used herein typically denotes humans, but may also encompass reference to non-human animals, preferably warm-blooded animals, more preferably mammals, such as, e.g., non-human primates, rodents, canines, felines, equines, ovines, porcines, and the like.

Conditions and diseases of particular relevance in the present invention include in particular acute heart failure (AHF), chronic heart failure (CHF) and sepsis.

In embodiments, the present predictive, prognostic and/or diagnostic methods for AHF or CHF may be used in individuals who have not yet been diagnosed as having AHF or CHF (for example, preventative screening), or who have been diagnosed as having AHF or CHF by the present or other means, or who are suspected of having AHF or CHF (for example, display one or more symptoms characteristic of AHF or CHF), or who are at risk of developing AHF or CHF (for example, genetic predisposition; presence of one or more developmental, environmental or behavioural risk factors). The methods may also be used to detect various stages of progression or severity of AHF or CHF. The methods may also be used to detect response of AHF or CHF to prophylactic or therapeutic treatments or other interventions.

In embodiments, the present predictive, prognostic and/or diagnostic methods for sepsis may be used in individuals who have an infection, or have sepsis, but who have not yet been diagnosed as having sepsis, or who are suspected of having sepsis, or who are at risk of developing sepsis. The methods may also be used to detect various stages of progression or severity of sepsis, such as sepsis, severe sepsis, septic shock, and organ failure. The methods may also be used to detect response of sepsis to prophylactic or therapeutic treatments or other interventions.

“Sepsis” may be broadly characterised as encompassing initial systemic inflammatory response syndrome (SIRS), sepsis, severe sepsis (sepsis with acute organ dysfunction), septic shock (sepsis with refractory arterial hypotension), multiple organ dysfunction or failure and death.

“SIRS” is a systemic inflammatory response syndrome with no signs of infection. It can be characterized by the presence of at least two of the four following clinical criteria: fever or hypothermia (temperature >100.4° F. [38° C.] or <96.8° F. [36° C.,]), tachycardia (>90 beats per minute), tachypnea (>20 breaths per minute or PaCO2 <4.3 kPa [32 mm Hg] or the need for mechanical ventilation), and an altered white blood cell count of >12,000 cells/mm³ or <4000 cells/mm³, or the presence of >10% band forms, respectively.

“Sepsis” can be defined as SIRS with an infection. Infection can be diagnosed by standard textbook criteria or, in case of uncertainty, by an infectious disease specialist.

“Severe sepsis” can be defined as the presence of sepsis and at least one of the following manifestations of inadequate organ perfusion or function: hypoxemia (PaO2 <10 kPa [75 mm Hg]), metabolic acidosis (pH<7.30), oliguria (output <30 mL/hr), lactic acidosis (serum lactate level >2 mmol/L), or an acute alteration in mental status without sedation (i.e., a reduction by at least 3 points from baseline value in the Glasgow Coma Score).

“Septic shock” can be defined as the presence of sepsis accompanied by a sustained decrease in systolic blood pressure (<90 mm Hg, or a drop of >40 mm Hg from baseline systolic blood pressure) despite fluid resuscitation, and the need for vasoactive amines to maintain adequate blood pressure.

As many organisms can be the cause of sepsis, diagnosis often takes time and requires testing against panels of possible agents. Sepsis can also arise in many different circumstances and therefore sepsis can be further classified for example: incarcerated sepsis which is an infection that is latent after the primary lesion has apparently healed but may be activated by a slight trauma; catheter sepsis which is sepsis occurring as a complication of intravenous catheterization; oral sepsis which is a disease condition in the mouth or adjacent parts which may affect the general health through the dissemination of toxins; puerperal sepsis which is infection of the female genital tract following childbirth, abortion, or miscarriage; sepsis lenta, which is a condition produced by infection with a-hemolytic streptococci, characterized by a febrile illness with endocarditis.

For the purposes of this invention, the term “sepsis” encompasses all above described forms and conditions in any stages of the disease progression.

The term “heart failure” broadly refers to a pathological condition characterised by an impaired diastolic or systolic blood flow rate and thus insufficient blood flow from the ventricle to peripheral organs.

“Acute heart failure” is defined as the rapid onset of symptoms and signs secondary to abnormal cardiac function. It may occur with or without previous cardiac disease. The cardiac dysfunction may be related to systolic or diastolic dysfunction, to abnormalities in cardiac rhythm, or to preload and afterload mismatch. It is often life threatening and requires urgent treatment. AHF can present itself acute de novo (new onset of acute heart failure in a patient without previously known cardiac dysfunction) or as acute decompensation of CHF.

According to established classification, AHF includes several distinct clinical conditions of presenting patients: (I) acute decompensated congestive heart failure, (II) AHF with hypertension/hypertensive crisis, (III) AHF with pulmonary oedema, (IVa) cardiogenic shock/low output syndrome, (IVb) severe cardiogenic shock, (V) high output failure, and (VI) right-sided acute heart failure. For detailed clinical description, classification and diagnosis of AHF, and for summary of further AHF classification systems including the Killip classification, the Forrester classification and the ‘clinical severity’ classification, refer to Nieminen et al. 2005 (“Executive summary of the guidelines on the diagnosis and treatment of acute heart failure: the Task Force on Acute Heart Failure of the European Society of Cardiology'. Eur Heart J 26: 384-416) and references therein.

The terms “chronic heart failure” (CHF) or “congestive heart failure” as used herein mean a case of heart failure that progresses so slowly that various compensatory mechanisms work to bring the disease into equilibrium. Common clinical symptoms of CHF include inter alia any one or more of breathlessness, diminishing exercise capacity, fatigue, lethargy and peripheral oedema. Other less common symptoms include any one or more of palpitations, memory or sleep disturbance and confusion, and usually co-occur with one or more of the above recited common symptoms.

In our predictive, prognostic and/or diagnostic methods, the presence and/or quantity of one or more proBNP or NTproBNP fragments and C-terminally truncated forms thereof as disclosed herein, and optionally of one or more further biomarkers of interest, as determined from a sample of a tested subject, is compared to a respective healthy or disease reference, or to a healthy or disease reference profile. Healthy or disease references or reference biomarker profiles can be generated from one individual or from a population of individuals of the desired clinical status or picture (for example, healthy, at risk but non-symptomatic, symptomatic disease, presence of particular symptoms, a given degree of disease severity, etc.). Such population may comprise without limitation 2, 10, 100, or even several hundreds or more individuals.

For instance, suitable disease reference(s) or reference biomarker profile(s) for AHF or CHF may be determined from individuals who are AHF- or CHF-symptomatic, or from individuals with increased risk of developing AHF or CHF, etc. For instance, suitable disease reference(s) or reference biomarker profile(s) for sepsis may be determined from individuals who are sepsis-positive and suffering from one of the stages in the progression of sepsis, or from individuals with increased risk of developing sepsis, or from individuals who do not have SIRS, or from individuals who do not have SIRS but who are suffering from an infectious process, or from individuals who are suffering from SIRS without the presence of sepsis, or from individuals who are suffering from the onset of sepsis, etc.

The respective healthy reference(s) or reference biomarker profile(s) may be generated from a healthy population.

Hence, “indicative” of AHF, CHF or sepsis as used herein broadly denotes an indication of a particular phenotype of or within AHF, CHF or sepsis (for example, at risk but non-symptomatic, symptomatic disease, presence of particular symptoms, a given degree of disease severity, etc.) as present in the individual or group of individuals from whom the respective disease reference or disease reference biomarker profile has been established. Similarly, the term “having AHF, CHF or sepsis” as used herein may broadly denote subjects having a particular phenotype of or within AHF, CHF or sepsis (for example, at risk but non-symptomatic, symptomatic disease, presence of particular symptoms, a given degree of disease severity, etc.).

In an embodiment, the reference(s) or reference biomarker profile(s) and test value(s) or test biomarker profile(s) that are compared herein may also be generated from the same subject for the purposes of monitoring the subject's condition over time (i.e. at two different time points e.g. upon admission and dismissal of the hospital or before, during or after treatment, etc.). This can inter alia allow monitoring in said subject disease progression, disease aggravation or alleviation, disease recurrence, response to treatment, response to other external or internal factors, conditions, or stressors, etc.

The present methods comprise comparing test value(s) or test biomarker profile(s) with reference(s) or reference biomarker profile(s). Such comparison may generally include any means to determine the presence or absence of at least one difference between the values or profiles being compared. A comparison may include a visual inspection, an arithmetical or statistical comparison of measurements. Such statistical comparisons include, but are not limited to, applying a rule. If the values or biomarker profiles comprise at least one standard, the comparison to determine a difference in said values or biomarker profiles may also include measurements of these standards, such that measurements of the biomarker are correlated to measurements of the internal standards.

The term “altered” with reference to the presence and/or quantity of a particular analyte generally encompasses any direction (e.g., increase or decrease) and extent of such alteration. For example, an alteration may encompass a decrease in a given value, without limitation, a decrease by at least about 10%, or by at least about 20%, or by at least about 30%, or by at least about 40%, or by at least about 50%, or by at least about 60%, or by at least about 70%, or by at least about 80%, or by at least about 90%, relative to a value with which a comparison is being made. For example, an alteration may encompass an increase in a given value, without limitation, an increase by at least about 10%, or by at least about 20%, or by at least about 40%, or by at least about 60%, or by at least about 80%, or by at least about 100%, or by at least about 150% or 200% or even by at least about 500% or like, relative to a value with which a comparison is being made.

Preferably, an alteration may refer to a change which falls outside of error margins of reference values in a given population (as expressed, for example, by standard deviation or standard error, or by a predetermined multiple thereof, e.g., ±1×SD or ±2×SD, or ±1×SE or ±2×SE). Alteration may also refer to a value falling outside of a reference range defined by or ≧85% or ≧90% or ≧95% or even ≧100% of values in said population). Multi-parameter analyses may be employed mutatis mutandis to determine alterations between groups of values and profiles generated there from.

The term “comparable” with reference to values and profiles is broadly used herein for situations when upon comparison a skilled person would not conclude an alteration, more preferably not a significant alteration.

In view of the above, the invention also contemplates inhibitor(s) of dipeptidyl-peptidase IV (DPPIV) and/or an inhibitor of Leu-aminopeptidases that may be responsible for the cleavage of the first 2, 3 or 6 N-terminal amino acids or more from the NTproBNP or proBNP proteins (i.e. resulting in 3-108, 4-108, or 7-108 proBNP fragments or in 3-76, 4-76, or 7-76 NTproBNP fragments) for treating AHF, CHF or sepsis; or a pharmaceutical composition comprising an inhibitor of said DPPIV and/or Leu-aminopeptidase for treating AHF, CHF or sepsis and the use of an inhibitor of said DPPIV and/or Leu-aminopeptidase for the manufacture of a medicament for treating AHF, CHF or sepsis. In addition, the invention provides method for treating AHF, CHF or sepsis in a subject in need of such treatment, comprising administering to said subject a therapeutically or prophylactically effective amount of an inhibitor of said DPPIV and/or Leu-aminopeptidase. Such inhibitors may be administered and/or may be in a composition for combined administration, simultaneously, separately or sequentially in any order.

The invention also contemplates the herein disclosed novel proBNP and NTproBNP fragments and C-terminally truncated forms thereof and uses therefore. Such peptides may be particularly suited, without limitation, as positive controls, standards or calibrators in the assays, prognosis and diagnosis methods, and kits of the invention. Such peptides may also be used as binders for or positive controls, standards or calibrators for binding of specific-binding agents directed thereto. The peptides may be supplied in any form, inter alia as precipitate, vacuum-dried, lyophilisate, in solution as liquid or frozen, or covalently or non-covalently immobilised on solid phase, such as for example, on solid chromatographic matrix or on glass or plastic or other suitable surfaces (e.g., as a part of peptide arrays and microarrays). The peptides may be readily prepared, for example, isolated from natural sources, or prepared recombinantly or synthetically.

Further, the herein disclosed novel proBNP and NTproBNP fragments and C-terminally truncated forms thereof may be labelled. The term “label” as used throughout this specification refers to any atom, molecule, moiety or biomolecule that can be used to provide a detectable and preferably quantifiable read-out or property, and that can be attached to or made part of an entity of interest, such as a peptide or polypeptide or a specific-binding agent. Labels may be suitably detectable by mass spectrometric, spectroscopic, optical, colorimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes alone or in combination with moieties that can suppress or shift emission spectra by fluorescence resonance energy transfer (FRET).

In an embodiment, the herein disclosed novel proBNP and NTproBNP fragments and C-terminally truncated forms thereof may be labelled by a mass-altering label.

Preferably, a mass-altering label may involve the presence of a distinct stable isotope in one or more amino acids of the peptide vis-à-vis its corresponding non-labelled peptide. Mass-labelled peptides are particularly useful as positive controls, standards and calibrators in mass spectrometry applications. In particular, peptides including one or more distinct isotopes are chemically alike, separate chromatographically and electrophoretically in the same manner and also ionise and fragment in the same way. However, in a suitable mass analyser such peptides and optionally select fragmentation ions thereof will display distinguishable m/z ratios and can thus be discriminated.

Examples of pairs of distinguishable stable isotopes include H and D, ¹²C and ¹³O, ¹⁴N and ¹⁵N or ¹⁶O and ¹⁸O. Usually, peptides and proteins of biological samples analysed in the present invention may substantially only contain common isotopes having high prevalence in nature, such as for example H, ¹²O, ¹⁴N and ¹⁶O. In such case, the mass-labelled peptide may be labelled with one or more uncommon isotopes having low prevalence in nature, such as for instance D, ¹³C, ¹⁵N and/or ¹⁸O. It is also conceivable that in cases where the peptides or proteins of a biological sample would include one or more uncommon isotopes, the mass-labelled peptide may comprise the respective common isotope(s).

Isotopically-labelled synthetic peptides may be obtained inter alia by synthesising or recombinantly producing such peptides using one or more isotopically-labelled amino acid substrates, or by chemically or enzymatically modifying unlabelled peptides to introduce thereto one or more distinct isotopes. By means of example and not limitation, D-labelled peptides may be synthesised or recombinantly produced in the presence of commercially available deuterated L-methionine CH₃—S-CD₂CD₂-CH(NH₂)—COOH or deuterated arginine H₂NC(═NH)—NH—(CD₂)₃—CD(NH₂)—COOH. It shall be appreciated that any amino acid of which deuterated or ¹⁵N- or ¹³C-containing forms exist may be considered for synthesis or recombinant production of labelled peptides. In another non-limiting example, a peptide may be treated with trypsin in H₂¹⁶O or H₂¹⁸O, leading to incorporation of two oxygens (¹⁶O or ¹⁸O, respectively) at the COOH-termini of said peptide (e.g., US 2006/105415). Further examples of such labelled reference peptides can be found in European Patent Application published as EP 1 634 083 A2 and in United States Patent Application published as US-2007-0254371-A1 both in the name of Pronota, which are hereby incorporated by reference in their entirety.

Addition of a known amount such labelled reference peptides as indicated above to the sample to be analyzed will upon performing Mass-Spectrometry analysis, result in a dual peak: one corresponding to the labelled (i.e. mass-altered) synthetic reference peptide and one corresponding to the target peptide present in the sample. Determining the surface of peaks and calculating their ratio will, given that the exact amount of the labelled reference peptide is known, lead to accurate calculation of the amount of target protein in the sample.

In a further embodiments of the invention, a kit is therefore provided comprising synthetic reference peptides for use in the quantification of target BNP-fragments of the invention, i.e. the proBNP fragments 3-108, 4-108 and 7-108, or a C-terminally truncated form of any one thereof and/or the NTproBNP fragments 3-76, 4-76 and 7-76, or a C-terminally truncated form of any one thereof. In a preferred embodiment, said reference peptides are selected from the group of: fragments 3-21 (SEQ ID NO: 6), 4-21 (SEQ ID NO: 7) or 7-21 (SEQ ID NO: 8) of a proBNP or NTproBNP peptide, such as for example the human proBNP or NTproBNP peptides shown in FIGS. 1B and C, respectively. Said reference peptides are preferably labelled or mass-altered as described herein.

In yet a further embodiment of the invention, methods are provided for the quantitative detection of a specific proBNP or NTproBNP fragment selected from the group of proBNP fragments 3-108, 4-108 and 7-108, or a C-terminally truncated form of any one thereof and/or the NTproBNP fragments 3-76, 4-76 and 7-76, or a C-terminally truncated form of any one thereof in a sample of a subject, comprising the steps of:

a) adding to the sample a known amount of any one of the reference peptides of the invention, that are labelled or mass-altered;
b) performing Mass-Spectrometric analysis of the sample;
c) determining the surface of the peak (1) corresponding to the reference peptide;
d) determining the surface of the peak (2) corresponding to the target peptide;
e) calculating the ratio of peaks 1 and 2; and
f) calculating the exact amount of the target protein, based on the ratio of step e) and the known amount of reference peptide added to the sample in step a).

Preferably, said reference peptides are isolated fragments of pro-B-type natriuretic peptide (proBNP) selected from proBNP 3-108, 4-108 and 7-108, or a C-terminally truncated form of any one thereof, and/or NTproBNP selected from NTproBNP 3-76, 4-76 and 7-76, or any C-terminally truncated form of any one thereof as explained above. More preferably, said proBNP or NTproBNP is human. In a more preferred embodiment, the C-terminally truncated form of said fragments of proBNP or NTproBNP is obtainable or directly obtained by proteolytic fragmentation of the proBNP or NTproBNP fragment, e.g. by proteolysis with an endopeptidase as explained above, most preferably by trypsin. In a highly preferred embodiment, said reference peptide is selected from the group of proBNP or NTproBNP 3-21, 4-21 and 7-21. Preferably, said reference peptides are labelled or mass-altered as described herein.

Said kits of the invention are preferably used and/or usable in the methods of diagnosis, prognosis and prediction of disease conditions or disorders which can be measured based on BNP and/or proBNP and/or NTproBNP activity or protein levels, such as acute (AHF) or chronic heart failure (CHF) and sepsis, encompassing initial systemic inflammatory response syndrome (SIRS), sepsis, severe sepsis (sepsis with acute organ dysfunction), septic shock (sepsis with refractory arterial hypotension), multiple organ dysfunction or failure and death, as described herein.

The invention further provides specific-binding agents as taught herein, optionally comprising label(s) as defined herein. The term “aptamer” refers to single-stranded or double-stranded oligo-DNA, oligo-RNA or oligo-DNA/RNA or any analogue thereof, which can specifically bind to a target molecule such as a peptide. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

Also provided are methods for immunising animals, e.g., non-human animals such as laboratory or farm, animals using (i.e., using as the immunising antigen) the herein disclosed novel proBNP and NTproBNP fragments and C-terminally truncated forms thereof, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds and mammals. Alternatively, sharks may also be used. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse.

The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Immune sera obtained or obtainable by immunisation as taught herein may be particularly useful for generating antibody reagents that specifically bind to one or more of the novel proBNP or NTproBNP fragments and C-terminally truncated forms disclosed herein.

As noted, the invention also teaches a method for selecting specific-binding agents which bind (a) one or more of the novel proBNP or NTproBNP fragments and C-terminally truncated forms as disclosed herein, substantially to the exclusion of (b) other proBNP and NTproBNP fragments. Preferably, the binding agents are specific for the NTproBNP 3-76 and/or 4-76 or proBNP 3-108 and/or 4-108 fragments and C-terminally truncated forms thereof or are insensitive for the NTproBNP 7-76 and/or proBNP 7-108 fragment and further N-terminally and C-terminally truncated fragments thereof. Conveniently, such methods may be based on subtracting or removing binding agents which cross-react or cross-bind the non-desired proBNP and NTproBNP peptides under (b). Such subtraction may be readily performed as known in the art by a variety of affinity separation methods, such as affinity chromatography, affinity solid phase extraction, affinity magnetic extraction, etc.

The invention also provides kits for predicting, prognosing and/or diagnosing AHF, CHF or sepsis in a subject. In an embodiment, such kits comprise at least one of the herein disclosed novel proBNP or NTproBNP fragments and/or C-terminally truncated forms thereof, and optionally in addition one or more other biomarkers useful in prognosing or diagnosing AHF, CHF or sepsis. In another embodiment, such kits comprise at least one specific-binding agent for one or more of the herein disclosed novel proBNP or NTproBNP fragments and C-terminally truncated forms thereof; specific-binding agents for other biomarkers relevant in AHF, CHF or sepsis may optionally be included in addition. In an embodiment, the kit may comprise a combination of both. In a further embodiment, the peptides and/or binding agents included in said kit may be labelled as taught herein.

The peptides, polypeptides and biomarkers in a kit may be part of an array, or they may be packaged separately and/or individually. The kit may also comprise at least one standard to be used in generating the biomarker profiles of the present invention. The kits of the present invention also may contain specific-binding agents that can be used to detectably label biomarkers contained in the biological samples from which the biomarker profiles are generated. The specific-binding agent(s) in a kit may be free or immobilised to a solid phase, may be part of an array, multi-well plate or they may be packaged separately and/or individually. Said kits may be particularly suitable for performing the assay methods of the invention, such as, e.g., immunoassays, ELISA assays, mass spectrometry assays, and the like.

Once a condition of AHF, CHF or sepsis has been diagnosed, the identification of the biomarkers of the present invention could be of use in the treatment or amelioration of the sepsis condition of the subject.

It is possible to increase the expression level or abundance of a peptide or polypeptide in a subject by administrating such a purified, synthetically or recombinantly produced biomarker of the invention to a subject having a reduced level of said biomarker in comparison to a healthy subject. Administering agents that increase the expression or activity of said biomarker may also be beneficial to the patient.

Another possibility can be the reduction of the level or abundance of a certain biomarker of the invention in case said biomarker has an increased occurrence in the blood of patients having AHF, CHF or sepsis when compared to healthy patients. Administering agents that reduce the expression or activity of said proteins may be beneficial to the subject.

The above aspects and embodiments are further supported by the following non-limiting examples.

### Example 1

### AHF, BNP-Processing Initial Experimental Observations

In a first experiment (Experiment 1), the sample used for analysis was a pool of 2 plasma samples obtained from 2 individuals upon hospital admission and at the time diagnosed with acute heart failure (AHF).

The plasma samples were depleted for the 14 most abundant proteins using an Agilent Multiple Affinity Removal System column (MARS Human-14, Agilent Technologies, Palo Alto, Calif., US). Depletion efficiency was checked using ELISA's and Western Blot analysis. Following depletion the 2 samples were pooled. Subsequently the sample was prepared for MASStermind analysis according the standard N-ter COFRADIC procedures. The COFRADIC sorting was performed on a peptide load corresponding 500 μg of depleted and processed protein material, as determined by BCA (Pierce, Rockford, Ill., US) prior tryptic digestion. The COFRADIC sorting was performed with TFA-based mobile phases and the 12 sorted fractions were automatically re-distributed in 32 aliquots. After drying these 32 fractions were reconstituted in an aqueous formic acid buffer (44 μL) and further analysed by a highly performant reversed phase (RP) nano-LC separation coupled on line with a QqTOF mass spectrometer via an electrospray ionisation (ESI) interface. The setup involved a classic column switching setup comprising a 300 μm i.d.×5 mm C18 RP precolumn and a custom made 100 μm i.d.×120 cm analytical nano RP-column. The mass spectrometer was operated in the information dependent analysis (IDA) mode. The IDA criteria adopted for precursor ion selection were: a m/z range of 300-1500; a 1s accumulation time and selection of the 2 most intense 2⁺ or 3⁺ charged signals per scan for fragmentation, if signal intensity exceeded a set threshold of 40 cps. Selected precursor ion masses were then excluded for 300 s. For the product ions spectra acquisitions at m/z range of 70-1500 was set. Optimal collision energy values were automatically determined and MS/MS accumulation was set to 3 s. Mass spectrometric data was collected during the entire nano-LC run. To retract the maximum from the samples all 32 aliquots were analyzed in duplo: during the re-analysis the precursor masses already selected for MS/MS in the first run were excluded for fragmentation in the 2^{nd} run, enabling a more in depth mining of the proteome. The collected MS/MS spectral data were then searched against the Swiss-Prot database using the standard COFRADIC parameterization. Within this dataset, 2 different tryptic peptides were assigned to the protein Natriuretic peptides B [Precursor] (Swiss-Prot entry P16860; ANFB_HUMAN). Compliant the COFRADIC principle the retrieved peptides were acetylated, which means the peptides correspond to in vivo available protein N-termini. In Table 1 the respective sequences as well as the associated Mascot scores are presented. The latter scores obviously demonstrate—to the skilled person in the field—the validity of the hits. More precisely two N-terminally processed forms of the same N-terminus, i.e., the N-terminus minus the 2 first N-terminal amino acids and the N-terminus minus the 6 first N-terminal amino acids were identified.

As mentioned, the fact the retrieved peptides are acetylated implicates an underlying biological activity, i.e., in vivo cleavage. Without being limited to any hypothesis, the here reported NT-ProBNP/ProBNP processing could agree with 2 consecutive dipeptidyl-peptidase IV (DPPIV) dipeptide cleavage events (selective cleavage after XXX-proline and XXX-glycine; cf. Table 1), disclosing novel biological insights into the role ANFB_HUMAN in heart failure.

In a second experiment (Experiment 2), the sample used for analysis consisted of a 1:1 mixture of 2 plasma pools—hereafter called pool A and pool B—whereby pool A contained plasma samples from patients with acute heart failure (AHF) at admission to hospital and pool B is derived from the same 8 patients after x days of treatment in hospital and clinically proven to be recovered. The selected AHF patients were included only if they had a history of chronic heart failure with previous decompensation, a low ejection fraction as measured by echocardiography and if they had high BNP values, as determined by a commercial assay. Inclusion criteria for the CHF group were chronic stable disease as defined by clinical parameters and low ejection fraction by echocardiography.

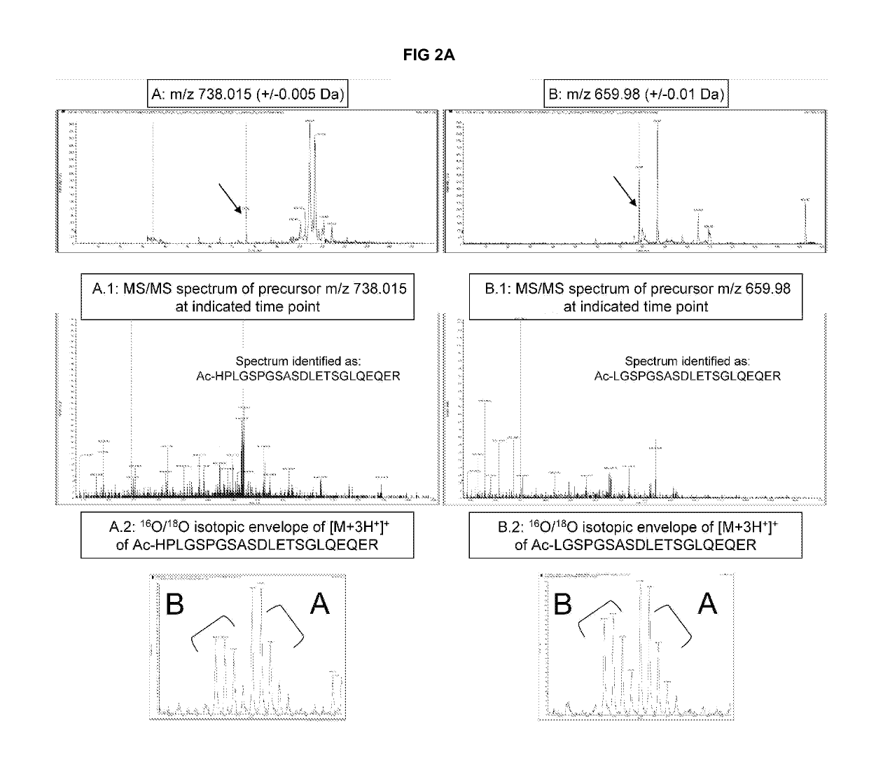

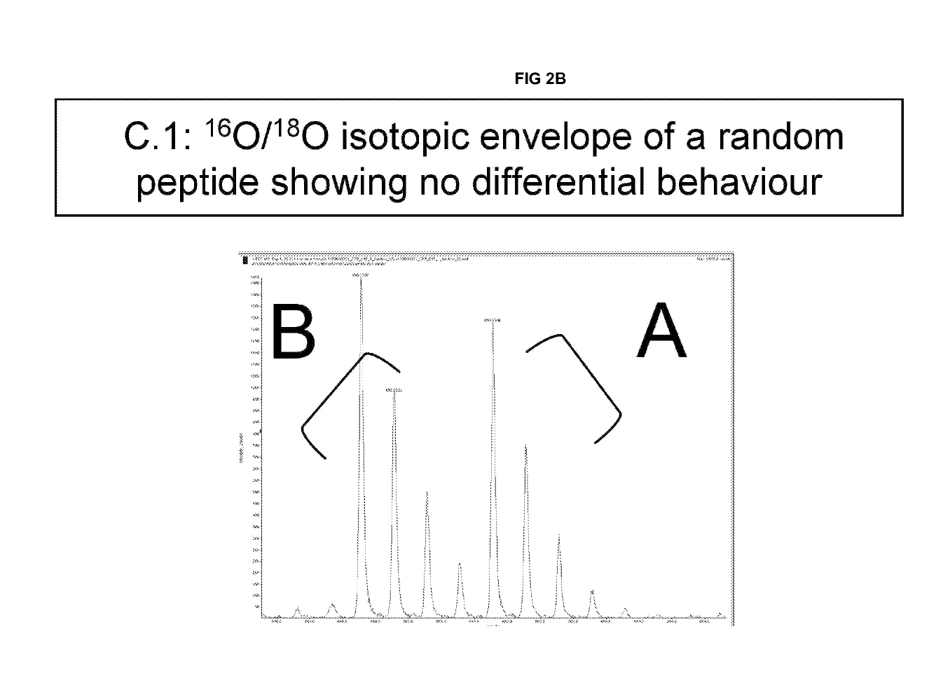

All 16 plasma samples were again depleted as mentioned under the first experiment. After this the 2 pools, each constituting 8 samples, were prepared. The 2 pools were further prepared for MASStermind analysis according the standard N-ter COFRADIC procedures. Then the respective pools were differentially labelled by trypsin mediated incorporation of ¹⁸O/¹⁶O, whereby pool A carried the heavy oxygen label and pool B ¹⁶O. Different from experiment 1 the COFRADIC sorting involved a 350 μg load (175 μg pool A+175 μg pool B) and was performed with NaOAc-based mobile phases in order no to compromise the label integrity. Again the 12 sorted fractions were automatically re-distributed in 32 aliquots. The further nano-LC-ESI-MS/MS analysis was similar compared to experiment 1; again duplicate measurements were executed. Within this dataset, 2 NT-ProBNP/ProBNP peptides were identified i.e. the known true N-terminus of NT-ProBNP/ProBNP and the N-terminus minus the 2 first N-terminal amino acids which also retrieved in experiment 1 (Table 1). Due to the differential nature of the experiment preliminary ratio readouts could be derived from the LC-MS data. FIG. 2 summarizes some exemplary data (raw data) derived from Example 1, experiment 2. The traces A and B depict the extracted ion chromatograms at m/z 738.015 (+/−0.005Da) and 659.98 (+/−0.01Da) which respectively agree the m/z-values of the ¹⁸O-labelled triply charged [M+3H⁺]³⁺ sequences (A) N-terminal acetylated-HPLGSPGSASDLETSGLQEQER (SEQ ID NO: 5) and (B) N-terminal acetylated-LGSPGSASDLETSGLQEQER (SEQ ID NO: 6). The latter tryptic peptides correspond to the true N-terminus of NT-ProBNP/ProBNP (up to the first tryptic cleavage site as indicated by the C-terminal arginine (R)) and the N-terminus of NT-ProBNP/ProBNP minus the 2 first amino acids (again up to the first tryptic cleavage site as indicated by the C-terminal arginine (R)). The exact elution times of the peptides of interest is indicated by the arrows and is confirmed by inspection of the corresponding MS/MS spectra: At the given time points (cf. arrows) the [M+3H⁺]³⁺ precursor masses were selected by the mass spectrometer for fragmentation, leading to the respective raw MS/MS spectra as depicted in A.1 and B.1. The latter spectra where positively identified by database searching (Mascot™ search algorithm, Matrix Science Inc., Boston, Mass., US) as being respectively acetylated-HPLGSPGSASDLETSGLQEQER (A.1) and acetylated-LGSPGSASDLETSGLQEQER (A.2). The images A.2, B.2 and C.1 depict the ¹⁶O/¹⁸O isotopic envelopes observed for the ¹⁶O-labelled-[M+3H⁺]³⁺ (pool B) and ¹⁸O-labelled-[M+3H⁺]³⁺ (pool A) signals of (A.2) acetylated-HPLGSPGSASDLETSGLQEQER, (B.2) acetylated-LGSPGSASDLETSGLQEQER and (C.1) a random peptide demonstrating no differential behaviour. The slight offset of the expected 1:1 ratio in image C.1 is due to imperfect 1:1 mixing. This offset falls well within the criteria of experimental variability and is normally corrected for during data processing.

### Example 2

### Sepsis, BNP-Processing Initial Experimental Observations

In a third experiment (Experiment 3), the sample used for analysis was a pool of plasma samples obtained from 9 individuals diagnosed with sepsis (post operation).

The plasma samples were depleted for the 12 most abundant proteins using an Genway_human depletion column (Beckman via Amersham Biosciences, Uppsala, Sweden). Depletion efficiency was checked using ELISA's and Western Blot analysis. Following depletion the 9 samples were pooled. Subsequently the sample was prepared for MASStermind analysis according the standard N-ter COFRADIC procedures. The COFRADIC sorting procedure applied was an adopted version of the high temperature/long column variant as described in Journal of Separation Science, Vol. 30, p 658-668, 2007 by Sandra et al. A peptide load corresponding 800 μg of depleted and processed protein material, as determined by BCA (Pierce, Rockford, Ill., US) prior tryptic digestion was used. The COFRADIC sorting was performed with NH4OAc-based mobile phases and the sorted fractions were collected in 4×384 well plates (1184 wells used). The latter were dried and the content of all wells was manually re-distributed in 60 aliquots of similar peptide mass, based on an in-house developed pooling protocol. After drying these 60 fractions were reconstituted in an aqueous formic acid buffer (120 μL) and further analysed by a reversed phase (RP) nano-LC separation coupled on line with a QqTOF mass spectrometer via an electrospray ionisation (ESI) interface. The setup involved a classic column switching setup comprising a 300 μm i.d.×5 mm C18 RP precolumn and 75 μm i.d.×15 cm analytical nano RP-column. The mass spectrometer was operated in the information dependent analysis (IDA) mode as described under experiment 1; again duplicate analyses were conducted. Within this dataset, also three different NT-ProBNP/ProBNP peptides were identified; i.e. the known true N-terminus of NT-ProBNP/ProBNP and the N-terminus minus the 2 first N-terminal amino acids and a novel N-terminus minus the 3 first N-terminal amino acids peptide (Table 1), pointing once more to a different in vivo processing of the NT-ProBNP/ProBNP.

Without being limited to any hypothesis, whilst the NT-ProBNP/ProBNP minus 2 is again suggestive for DPPIV involvement, the peptide missing the leucine at position 3 might be the result of Leu-aminopeptidase activity.

TABLE 1

| | | True N-terminus | True N-terminus | |
|---|---|---|---|---|
| | True N-terminus: | minus 2 AA | minus 3 AA | True N-terminus |
| | AA: 1-21 | AA: 3-21 | AA: 4-21 | minus 6 AA |
| | Ac- | Ac- | Ac- | AA: 7-21 |
| | HPLGSPGSASDLETSG | LGSPGSASDLETSGLQE | GSPGSASDLETSGLQE | Ac- |
| | LQEQER | QER | QER | GSASDLETSGLQEQER |
| Exp. 1 | — | 83.56 | — | 125.27 |
| Exp. 2 | 47.81 | 65.28 | — | — |
| (cf. FIG. 2) | | | | |
| Exp. 3 | 61.98 | 72.84 | 40.59 | — |

### Example 3

### Analysis of Patient Samples for the Presence of the Three Identified Fragments and their Relevance for Diagnosis, Prognosis or Prediction of BNP-Related Diseases

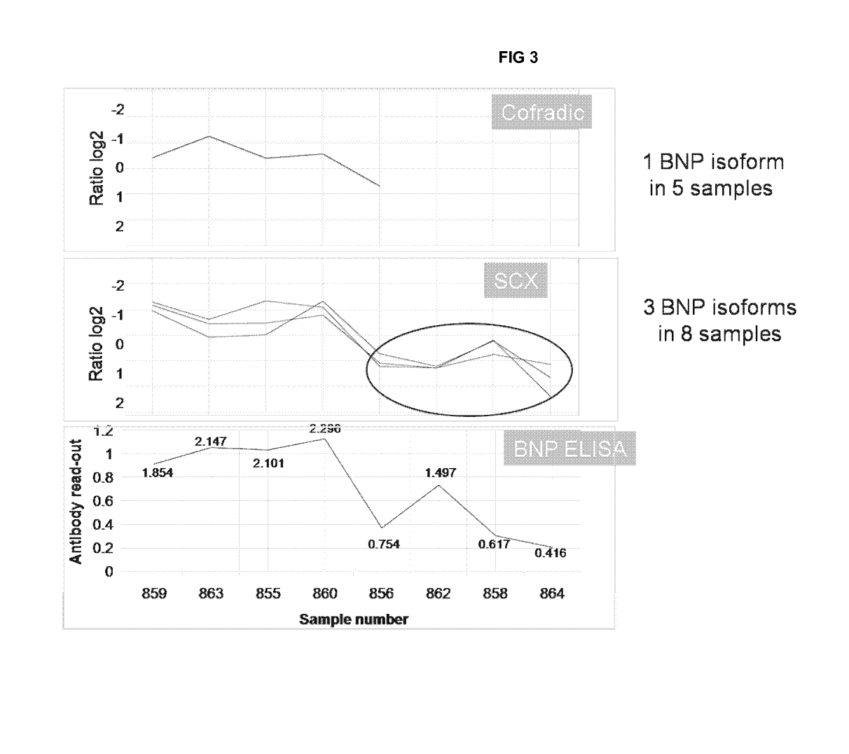

As shown in FIG. 3, we compared three different measurement methods of BNP: The known Cofradic™ method revealing one BNP isoform, which is undetectable under the detection threshold of +/−1 ng/ml (top panel), an improved SCX (strong cation exchange) column-based mass-spectrometry method developed by the inventors as explained below, revealing three different isoforms of BNP, detectable at the sub-nanogram level (middle panel) and a standard ELISA detection method commonly used in clinical settings, not able to distinguish between different proBNP or NTproBNP isoforms (lower panel).

The following describes the experimental parameters for the operation of a single step sorting platform in a reference design mode based on SCX isolation of N-terminal peptides, enabling the detection and quantification of the three different proBNP or NTproBNP fragments of the invention.

### Preparation of Samples in the Reference Design Mode:

2.048 ml of a reference plasma pool was depleted (8 depletion batches of 8 depletion runs, i.e. 64 depletion runs in total) and 224 μl of each individual crude plasma sample (7 depletion runs for each individual sample). Standard depletion includes 1:5 dilution of the crude sample material in buffer A, part of the MARS depletion system, spin filtering and depletion on the MARS depletion system (Agilent MARS Hu(14)). The flow through pools were subsequently concentrated 4× using a Vivaspin filter with a MWCO of 3000 Da to yield a protein concentration of 1 mg/ml for each flow through pool (concentration determined using BCA). Albumin and IgG depletion efficiency of each flow through pool was tested via Western Blotting. The flow throughs (64 in total) of the reference plasma pool were subsequently combined resulting in one reference plasma fraction and again divided for chemistry. The proteins were subsequently denatured by adding guanidinium hydrochloride (final concentration 3M), reduced and alkylated using TCEP and iodoacetamide added in a 25 and 50 molar excess, respectively. Reduction took place at 30° C. during 10 min; alkylation at 30° C. during 1 h. The mixtures were subsequently acetylated at 30° C. during 90 min by adding sulfo-N-hydroxysuccinimide-acetate in a 75 molar excess. A deacetylation, for 20 min at room temperature, with ammonium hydroxide in a 3.5 molar excess, against sulfo-NHS-acetate, was performed to deacetylate the serines and threonines that might get acetylated during the acetylation step. Following the deacetylation step, the samples were desalted on a PD10 column (Sephadex G-25 medium) and captured in a 2.5 mM NH₄HCO₃ buffer at pH 8. Protein recoveries were measured around 70% (BCA). The samples, present in 3.5 ml following PD-10 desalting and buffer exchange were subsequently dried to 500 μl and digested with trypsin in a substrate:trypsin ratio of 50:1 (w:w) by overnight incubation at 37° C. (no use of resuspension buffer). The samples were acidified to pH 6 (by adding 10% FA) the following day and completely dried. 150 μl of H₂¹⁶O was added to every reference plasma sample. 150 μl of H₂¹⁸O was added to every individual plasma sample. Labelling took place during 26 hrs. All reference plasma samples were again pooled after labelling and divided per 270 μg.

270 μg of each ¹⁸O labelled sample was combined with 270 μg ¹⁶O reference plasma sample in a controlled manner to prevent back-exchange. The mixing of both samples at pH 6 appeared to induce an immediate back-exchange. Therefore, the ¹⁶O and ¹⁸O labelled samples were acidified (to pH 3) prior to mixing. In this way back-exchange could be prevented. TFA (1%) was used to acidify the sample since FA interferes with the successful operation of the SCX column. After acidification ACN was added to a final concentration of 50%. The latter was used to prevent non-specific interaction with the SCX column. The final volume was 550 μl allowing the injection of 500 μl onto the SCX column. Injection of relative large volumes onto the SCX column allows the dilution of salts present in the sample; in the case presented salts originated from the NH₄HCO₃ buffer. If sample salt concentrations are too high, the binding of internal peptides onto the SCX column might be prevented. The final salt concentration in the sample was ˜15 mM which is not expected to interfere with the successful operation of the SCX column.

### SCX Operation:

The column used was a Zorbax 300 Angström SCX column (2.1 mm ID, 5 cm L, 3.5 μm particle diameter). Stationary phase consists of silica particles with negatively charged residues (sulfonic acid) attached. This residue is charged over a wide pH range. The 5 cm column has sufficient capacity to handle 500 μg (250 μg ¹⁶O and 250 μg ¹⁸O).

The SCX procedure consists of several steps:

(1) Sample loading using 10 mM sodium-phosphate (pH 3), 50% ACN. Flow-through is collected during this step in one glass vial (1.5 ml capacity with V-shape).
(2) Elution of the internal peptides in order to prepare the column for a next round of sorting. Two mobile phases are thereby used: 10 mM sodium-phosphate (pH 3), 20% ACN and 10 mM sodium-phosphate (pH 3), 20% ACN, 2M NaCl. The former is used to switch from 50% ACN containing buffer to 20% ACN containing buffer. The latter is used to elute the internal peptides. A NaCl gradient is thereby applied. By switching to 20% ACN containing buffers, salt precipitation is prevented.
(3) Second, short cleaning by injection of a KCl (2M) plug onto the column.
(4) Extensive equilibration of the column using 10 mM sodium-phosphate (pH 3), 50% ACN. This buffer is several times injected to equilibrate the injection system.

### 2D-Orthogonal Set-Up:

The SCX flow-through (1.3 ml) was completely dried. After re-dissolving the sample, it was injected onto a 2D orthogonal HPLC system to resolve the complex mixture of N-terminal peptides, C-terminal peptides and non-tryptic peptides. The 2D set-up consisted of a narrow-bore X-Terra Phenyl HPLC column (15 cm×2.1 mm ID×3.5 μm dₚ) and a nano C18 column (15 cm×75 μm×3 μm dₚ). The orthogonality of a phenyl and C18 column is limited when they are both operated at low pH. Therefore, the first dimension column is operated at high pH (pH 10). The X-Terra portfolio of columns is specifically designed for operation at higher pH. The nano-RPLC column was operated at pH 2. The SCX flow-through was dissolved in 530 μl 98% mobile phase A (10 mM NH₄OAc (pH 10)) and 2% mobile phase B (80% ACN, 10 mM NH₄OAc (pH 10)). The entire sample was subsequently injected onto the X-Terra Phenyl LC column. Large volume injection onto the column appeared to be feasible, which is of major importance to limit the sample loss during sample handling. A 60 min ACN gradient was applied to separate the mixture. 32 one minute fractions (100 μl volumes) were collected along the gradient. These fractions were dried and re-dissolved in 44 μl 0.1% FA; mobile phase A in the second dimension separation. 20 μl of all fractions were injected onto the nano-RPLC column and peptides were separated using a 60 min ACN gradient (mobile phase B: 80% ACN, 0.1% FA). During the separations, 260 spots were deposited on MALDI targets (15 sec spotting intervals). More than 8000 spots were generated using this set-up. All fractions were subsequently analyzed by MALDI-MS.

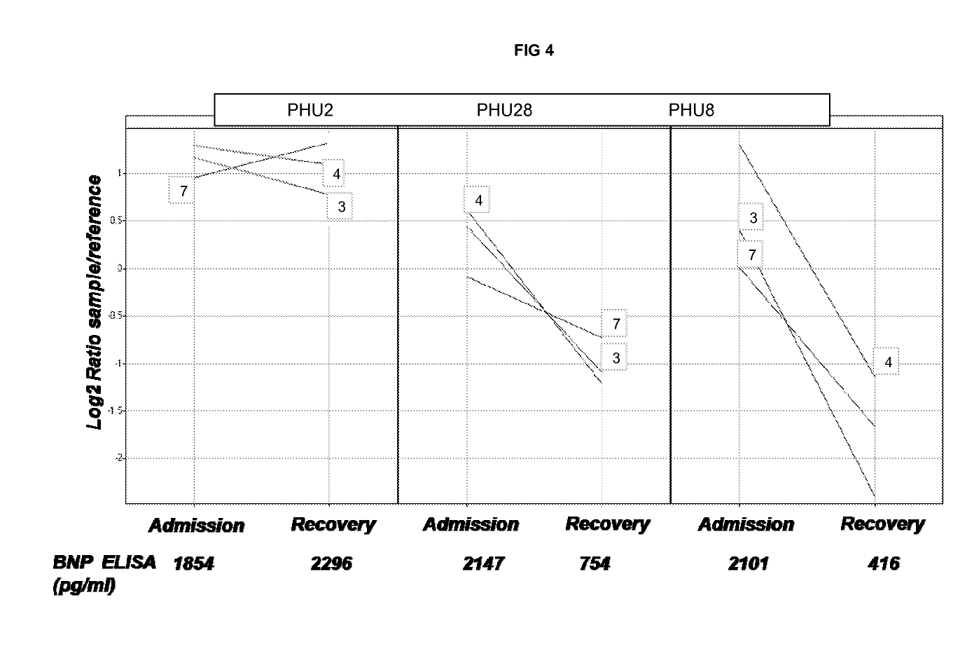

FIG. 4 shows the analysis of paired samples of patients, wherein each pair of samples is derived from the same patient, but at two different time points, upon admission to the hospital and upon discharge of the patient form the hospital after being treated for AHF and deemed clinically recovered. As can be seen from the figure, three NT-proBNP specific fragments, the NTproBNP 3-76, NTproBNP 4-76 and NTproBNP 7-76 can be detected in the samples using this SCX set-up. Overall the fragments show a similar trend, i.e. going down from admission to discharge, in patients Phu2, Phu28 and Phu8.

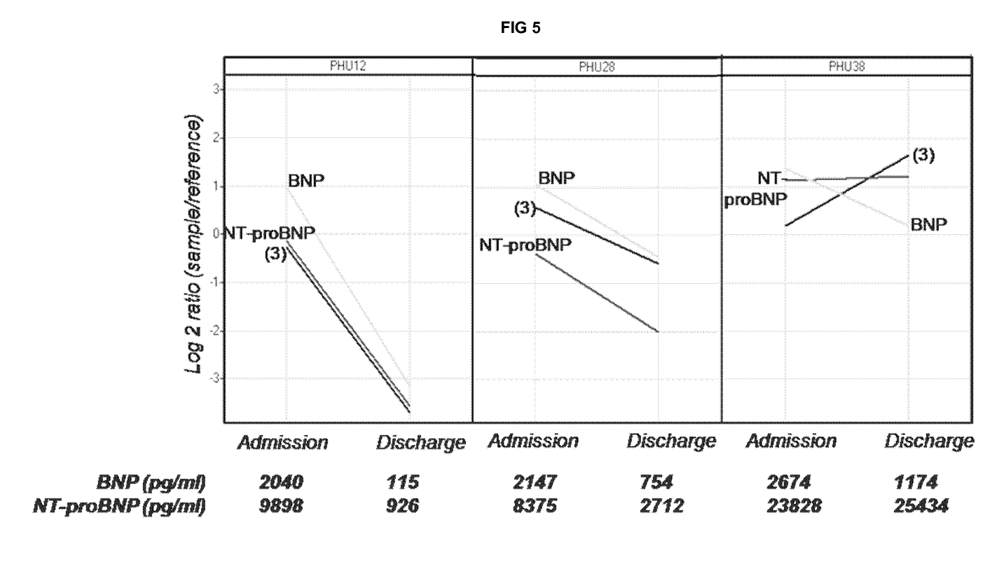

Being able to measure all the specific fragments allows to compare the profiles observed for each fragment with the ELISA measurements. The graphs in FIG. 5 represent the changes measured for BNP (Biosite assay), NTproBNP (Roche assay) and NTproBNP 3-76 (MS based) in 3 different patients. For patients Phu2 and Phu28 there is good correlation between both ELISA measurements and the MS based assay. However for patient Phu38 we observe a different trend for NTproBNP 3-76, i.e. going up, as opposed to the BNP ELISA outcome. In this patient there is also lack of good correlation between the BNP and the NTproBNP ELISA. Depending on the assay used a different outcome and hence associated clinical decision will be the result.

1-51. (canceled)

52. An isolated fragment of pro-B-type natriuretic peptide (proBNP) or amino terminal pro-B-type natriuretic peptide (NTproBNP) selected from: proBNP 3-108, 4-108 and 7-108, NTproBNP 3-76, 4-76 and 7-76, or a C-terminally truncated form of any one thereof.

53. The isolated fragment of proBNP or NTproBNP or C-terminally truncated form thereof according to claim 52, wherein said proBNP or NTproBNP is human.

54. The C-terminally truncated form of the fragment of proBNP or NTproBNP according to claim 52, obtainable or directly obtained by endoproteinase digest of the proBNP or NTproBNP fragment.

55. The C-terminally truncated form of the fragment of proBNP or NTproBNP according to claim 54, wherein the endoproteinase is trypsin.

56. The C-terminally truncated form of the fragment of proBNP or NTproBNP according to claim 52, selected from proBNP or NTproBNP 3-21, 4-21 and 7-21.

57. The isolated fragment of proBNP or NTproBNP or C-terminally truncated form thereof according to claim 52, further comprising a detectable label.

58. A method for assaying proBNP or NTproBNP in a sample, comprising specifically measuring the presence and/or quantity in said sample of one or more fragments of claim 52.

59. The method of claim 58 further comprising measuring the presence and/or quantity in said sample of proBNP 1-108 and C-terminally truncated forms thereof.

60. The method of claim 58 further comprising measuring the presence and/or quantity in said sample of NTproBNP 1-76 and C-terminally truncated forms thereof.

61. The method of claim 58, wherein the sample is from a human subject and the proBNP or NTproBNP is human.

62. The method of claim 58, which is an immunoassay method, a mass spectrometry analysis method or a chromatography method, or a combination thereof.

63. The method of claim 58, wherein the measurement is specific for the NTproBNP 3-76, 4-76 or proBNP 3-108 or 4-108 fragment, or C-terminally truncated fragments thereof and/or wherein the measurement is specific for NTproBNP or proBNP but insensitive for the proBNP 7-108 or NTproBNP 7-76 fragment or N- and/or C-terminally truncated fragments thereof.

64. A method for establishing a healthy reference or a disease reference for the presence and/or quantity of one or more fragments of proBNP, comprising:
(a) assaying proBNP in:
(aa) a sample from one or more subjects not having acute heart failure (AHF), chronic heart failure (CHF) or sepsis, or
(ab) a sample from one or more subjects having AHF, CHF or sepsis,
comprising specifically measuring the presence and/or quantity in said sample (aa) or (ab) of one or more fragments of proBNP selected from proBNP 3-108, 4-108 and 7-108 and C-terminally truncated forms thereof;
(b) storing the presence and/or quantity of the respective fragments of proBNP and C-terminally truncated forms thereof,
(ba) as measured in (aa) as a healthy reference or a healthy reference profile, or
(bb) as measured in (ab) as a disease reference or a disease reference profile.

65. A method for establishing a healthy reference or a disease reference for the presence and/or quantity of one or more fragments of NTproBNP, comprising:
(a) assaying NTproBNP in:
(aa) a sample from one or more subjects not having AHF, CHF or sepsis, or
(ab) a sample from one or more subjects having AHF, CHF or sepsis,
comprising specifically measuring the presence and/or quantity in said sample (aa) or (ab) of one or more fragments of NTproBNP selected from NTproBNP 3-76, 4-76 and 7-76 and C-terminally truncated forms thereof;
(b) storing the presence and/or quantity of the respective fragments of NTproBNP and C-terminally truncated forms thereof
(ba) as measured in (aa) as a healthy reference or a healthy reference profile, or
(bb) as measured in (ab) as a disease reference or a disease reference profile.

66. The method of claim 64, wherein the measurement is specific for the NTproBNP 3-76, 4-76 or proBNP 3-108 or 4-108 fragment, or C-terminally truncated fragments thereof and/or wherein the measurement is specific for NTproBNP or proBNP but insensitive for the proBNP 7-108 or NTproBNP 7-76 fragment or N- and/or C-terminally truncated fragments thereof.

67. The method of claim 65, wherein the measurement is specific for the NTproBNP 3-76, 4-76 or proBNP 3-108 or 4-108 fragment, or C-terminally truncated fragments thereof and/or wherein the measurement is specific for NTproBNP or proBNP but insensitive for the proBNP 7-108 or NTproBNP 7-76 fragment or N- and/or C-terminally truncated fragments thereof.

68. A method for the prediction, prognosis and/or diagnosis of AHF, CHF or sepsis in a subject, comprising:
(a) assaying proBNP in a sample from the subject, comprising specifically measuring the presence and/or quantity in said sample of one or more fragments of proBNP selected from proBNP 3-108, 4-108 and 7-108 and C-terminally truncated forms thereof;
(b) comparing the presence and/or quantity of said one or more fragments of proBNP and C-terminally truncated forms thereof as measured in (a):
(ba) with a respective healthy reference or healthy reference profile as defined in claim 65, whereby
(baa) a comparable presence and/or quantity of the proBNP fragments and C-terminally truncated forms thereof in the sample to the presence and/or quantity in the healthy reference or healthy reference profile is indicative of absence of AHF, CHF or sepsis, and/or
(bab) an altered presence and/or quantity of the proBNP fragments and C-terminally truncated forms thereof in the sample compared to the presence and/or quantity in the healthy reference or healthy reference profile is indicative of AHF, CHF or sepsis; and/or
(bb) with a respective disease reference or disease reference profile as defined in claim 65, whereby a comparable presence and/or quantity of the proBNP fragments and C-terminally truncated forms thereof in the sample to the presence and/or quantity in the disease reference is indicative of AHF, CHF or sepsis.

69. A method for the prediction, prognosis and/or diagnosis of AHF, CHF or sepsis in a subject comprising:
(a) assaying NTproBNP in a sample from the subject, comprising specifically measuring the presence and/or quantity in said sample of one or more fragments of NTproBNP selected from NTproBNP 3-76, 4-76 and 7-76 and C-terminally truncated forms thereof;
(b) comparing the presence and/or quantity of said one or more fragments of NTproBNP and C-terminally truncated forms thereof as measured in (a):
(ba) with a respective healthy reference or healthy reference profile as defined in claim 66, whereby
(baa) a comparable presence and/or quantity of the NTproBNP fragments and C-terminally truncated forms thereof in the sample to the presence and/or quantity in the healthy reference or healthy reference profile is indicative of absence of AHF, CHF or sepsis, and/or
(bab) an altered presence and/or quantity of the NTproBNP fragments and C-terminally truncated forms thereof in the sample compared to the presence and/or quantity in the healthy reference or healthy reference profile is indicative of AHF, CHF or sepsis; and/or
(bb) with a respective disease reference or disease reference profile as defined in claim 66, whereby a comparable presence and/or quantity of the NTproBNP fragments and C-terminally truncated forms thereof in the sample to the presence and/or quantity in the disease reference is indicative of AHF, CHF or sepsis.

70. The method of claim 68, wherein the measurement is specific for the NTproBNP 3-76, 4-76 or proBNP 3-108 or 4-108 fragment, or C-terminally truncated fragments thereof and/or wherein the measurement is specific for NTproBNP or proBNP but insensitive for the proBNP 7-108 or NTproBNP 7-76 fragment or N- and/or C-terminally truncated fragments thereof.

71. The method of claim 69, wherein the measurement is specific for the NTproBNP 3-76, 4-76 or proBNP 3-108 or 4-108 fragment, or C-terminally truncated fragments thereof and/or wherein the measurement is specific for NTproBNP or proBNP but insensitive for the proBNP 7-108 or NTproBNP 7-76 fragment or N- and/or C-terminally truncated fragments thereof.

72. A specific-binding agent capable of specifically binding to any one or more of the isolated fragments of proBNP or NTproBNP and C-terminally truncated forms thereof according to claim 52.

73. The specific-binding agent of claim 72 capable of specifically binding to only one of the isolated fragments of proBNP or NTproBNP and C-terminally truncated forms thereof according to claim 52.

74. The specific-binding agent according to claim 72, which is an antibody, aptamer, photoaptamer, protein, peptide, peptidomimetic or a small molecule.

75. The specific-binding agent according to claim 72, which is specific for the NTproBNP 3-76, 4-76 or proBNP 3-108 or 4-108 fragment, or C-terminally truncated fragments thereof and/or which specifically binds NTproBNP or proBNP but is insensitive for the proBNP 7-108 or NTproBNP 7-76 fragment or N- and/or C-terminally truncated fragments thereof.

76. A method of selecting the specific-binding agent according to claim 72 comprising:
(a) providing a plurality of test specific-binding agents;
(b) selecting from the test specific-binding agents of (a) specific-binding agents which bind to one or more desired isolated fragments of proBNP or NTproBNP and C-terminally truncated forms thereof selected from: proBNP 3-108, 4-108 and 7-108, NTproBNP 3-76, 4-76 and 7-76, or a C-terminally truncated form of any one thereof,
(c) removing from the specific-binding agents selected in (b) those which also bind to any of the not desired isolated fragments of proBNP or NTproBNP and C-terminally truncated forms thereof selected from: proBNP 3-108, 4-108 and 7-108, NTproBNP 3-76, 4-76 and 7-76, or a C-terminally truncated form of any one thereof and/or to proBNP 1-108 or NTproBNP 1-76 and C-terminally truncated forms thereof.

77. A kit comprising one or more isolated fragments of proBNP or NTproBNP or C-terminally truncated form thereof according to claim 52.

78. A kit comprising one or more specific-binding agents according to claims 72.

79. A method for immunising an animal using the isolated fragment of proBNP or NTproBNP or C-terminally truncated form thereof according to claim 52, optionally fused to or covalently or non-covalently linked, bound or adsorbed to a presenting carrier.

80. An immune serum or antibody reagent isolated from the animals immunised as in claim 79.

81. A method for predicting, diagnosing or prognosing recovery of sepsis or heart failure in a subject, comprising;
a) the measurement of one or more of the NTproBNP fragments 3-76 or 4-76, or BNP fragments 3-108 and 4-108 or C-terminally truncated forms thereof, at a first time point,
b) the measurement of one or more of the NTproBNP fragments 3-76 or 4-76, or BNP fragments 3-108 and 4-108 or C-terminally truncated forms thereof, at a later time point, and
c) comparing both values,
wherein a decrease in amount of either of the one or more fragments at the later time point as compared to the level at the first time point reflects recovery of heart failure of the patient and wherein an increase in amount of either of the one or more fragments in step b) compared to the level in step a) points towards a high risk of heart failure.

82. The method of claim 81, additionally comprising steps:
d) measuring the total amount of BNP or alternatively measuring the amount of NTproBNP 7-76 or BNP 7-108 or further N-terminal and/or C-terminally truncated forms thereof; and
e) calculating the ratio of the values obtained in steps a-b versus the value obtained in step d) in order to compensate for measurement errors.

83. The method of claim 81, wherein both fragments of NTproBNP 3-76 and 4-76 or BNP fragments 3-108 and 4-108 or C-terminally truncated forms thereof, are measured simultaneously.

84. A method for prognosis, diagnosis and or prediction of sepsis or heart failure in a subject comprising;
a) the measurement of one or more of the NTproBNP fragments 3-76 or 4-76, or BNP fragments 3-108 and 4-108 or C-terminally truncated forms thereof,
b) comparing the amount of the measurement in step a) to the level of said fragment(s) in a healthy subject,
wherein an increase in amount of either of the one or more fragments in the patient as compared to the healthy subject points towards a high risk of sepsis or heart failure.

85. The method of claim 84, wherein additionally the total amount of BNP or the amount of NTproBNP 7-76 or BNP 7-108 or C-terminally truncated forms thereof is measured and the ratio of a) the NTproBNP fragments 3-76 or 4-76, or BNP fragments 3-108 and 4-108 or C-terminally truncated forms thereof versus b) the total amount of BNP or the amount of NTproBNP 7-76 or BNP 7-108 or further N-terminal and/or C-terminally truncated forms thereof, is calculated in order to compensate for measurement errors.

86. The method of claim 84, wherein both fragments of NTproBNP 3-76 and 4-76 or BNP fragments 3-108 and 4-108 or C-terminally truncated forms thereof are measured simultaneously.

87. The method of claim 81, wherein said amount of NTproBNP does not include the 7-76 or 7-108 fragment or further N-terminal and/or C-terminally truncated forms thereof, and wherein an increase in the amount of NTproBNP points towards an increased risk of heart failure.

88. A method for assessing the risk of suffering from heart failure in a subject comprising;
a) measuring the total amount of NTproBNP in a sample, and
b) measuring specifically the fragment 7-76 of NTproBNP or 7-108 of BNP or further N-terminal and/or C-terminally truncated forms thereof,
c) calculating the total amount of NTproBNP in the sample minus the amount of the fragment 7-76 of NTproBNP or 7-108 of BNP or further N-terminal and/or C-terminally truncated forms thereof, and
d) establishing the difference between the measurement in step c) from the patient and the measurement in step c) from a healthy subject,
wherein an increase of said measurement in step c) in the patient as compared to a healthy subject indicates a high risk of suffering from heart failure and a decrease in said measurement in step c) in the patient as compared to a healthy subject indicates a low risk of suffering from heart failure.

89. The method of claim 88, wherein measurement is specifically insensitive to the NTproBNP fragment 7-76 or further N-terminal and C-terminal truncated forms thereof.

90. A method for assessing the risk of suffering from heart failure in a subject according to claim 89, wherein the measurement of NTproBNP is specifically insensitive to the NTproBNP fragment 7-76 or further N-terminal and C-terminal truncated forms thereof.

91. The method of claim 88, wherein measurement of NTproBNP in a sample, obtained by one or more selected from antibodies, aptamers, photoaptamers, proteins, peptides, peptidomimetics or a small molecules, characterised said measurement is specifically insensitive to the NTproBNP fragment 7-76 or further N-terminal and C-terminal truncated forms thereof.

92. The method of claim 58, wherein the quantification of said specific proBNP or NTproBNP fragment selected from the group of proBNP fragments 3-108, 4-108 and 7-108, or a C-terminally truncated form of any one thereof and/or the NTproBNP fragments 3-76, 4-76 and 7-76 or further N-terminal truncated forms of NTproBNP 7-76, or a C-terminally truncated form of any one thereof in a sample of a subject is done by Mass-spectrometry, comprising the steps of:
a) adding to the sample a known amount of any one of the reference peptides selected from: proBNP 3-108, 4-108 and 7-108, NTproBNP 3-76, 4-76 and 7-76, or a C-terminally truncated form of any one thereof, that are labelled or mass-altered;
b) performing Mass-Spectrometric analysis of the sample;
c) determining the surface of the peak (1) corresponding to the reference peptide;
d) determining the surface of the peak (2) corresponding to the target peptide;
e) calculating the ratio of peaks 1 and 2; and
f) calculating the exact amount of the target protein, based on the ratio of step e) and the known amount of reference peptide added to the sample in step a).


SEQUENCE LISTING (8 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 4 - protein, 32 aa, Homo sapiens
SPKMVQGSGCFGRKMDRISSSSGLGCKVLRRH

SEQ ID NO: 5 - protein, 22 aa, Artificial
  Synthetic peptide
HPLGSPGSASDLETSGLQEQER

SEQ ID NO: 6 - protein, 20 aa, Artificial
  Synthetic peptide
LGSPGSASDLETSGLQEQER

SEQ ID NO: 7 - protein, 19 aa, Artificial
  Synthetic peptide
GSPGSASDLETSGLQEQER

SEQ ID NO: 8 - protein, 16 aa, Artificial
  Synthetic peptide
GSASDLETSGLQEQER